SEQUENCE LISTING (13 sequences)

SEQ ID NO: 1 - protein, 1273 aa, severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 23 aa, severe acute respiratory syndrome coronavirus 2
GSFCTQLNRALTGIAVEQDKNTQ

SEQ ID NO: 3 - protein, 1273 aa, severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYHTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 4 - protein, 1273 aa, severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENLVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 5 - protein, 1273 aa, severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFAISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 6 - protein, 1273 aa, severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLFLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 7 - protein, 1273 aa, severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNLALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 8 - protein, 1273 aa, severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIALEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 9 - protein, 1273 aa, severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQQVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 10 - protein, 1273 aa, severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDCGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 11 - protein, 1273 aa, severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKSSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 12 - protein, 805 aa, Homo sapiens [sequence omitted]

SEQ ID NO: 13 - protein, 1273 aa, SARS-CoV-2; identical to SEQ ID NO: 1

(19) United States
(12) Patent Application Publication
(10) Pub. No.: US 2023/0346717 A1
(43) Pub. Date: Nov. 2, 2023

(54) IDENTIFICATION AND USE OF COMPOUNDS IN THE TREATMENT OR PREVENTION OF SEVERE ACUTE RESPIRATORY SYNDROME CORONAVIRUS 2

(71) Applicant: Vanda Pharmaceuticals Inc., Washington, DC (US)

(72) Inventors: Mihael H. POLYMEROPOULOS, Potomac, MD (US); Sandra SMIESZEK, Washington, DC (US); Bartlomiej PRZYCHODZEN, Washington, DC (US); Vasilios M. POLYMEROPOULOS, Potomac, MD (US)

(21) Appl. No.: 17/906,940

(22) PCT Filed: Mar. 26, 2021
(86) PCT No.: PCT/US2021/024304

§ 371 Date: Sep. 21, 2022

Related U.S. Application Data
(60) Provisional application No. 63/142,392, filed on Jan. 27, 2021.
(60) Provisional application No. 63/025,837, filed on May 15, 2020.
(60) Provisional application No. 63/025,631, filed on May 15, 2020.
(60) Provisional application No. 63/007,261, filed on Apr. 8, 2020.
(60) Provisional application No. 63/004,916, filed on Apr. 3, 2020.
(60) Provisional application No. 63/003,187, filed on Mar. 31, 2020.
(60) Provisional application No. 63/001,114, filed on Mar. 27, 2020.

(51) Int. Cl. A61K 31/13; A61P 31/14; G01N 33/50

(52) U.S. Cl. CPC A61K 31/13; A61P 31/14; G01N 33/5008; G01N 2333/96466; G01N 2333/165

(57) ABSTRACT

Methods for identifying compounds useful in treating or preventing infection by severe acute respiratory syndrome coronavirus 2 (SARS-CoV-2) are described, as are methods of treating or preventing SARS-CoV-2 infection using such compounds.

### CROSS-REFERENCE TO RELATED APPLICATIONS

This application claims the benefit of co-pending U.S. Provisional Patent Application Nos. 63/001,114, filed 27 Mar. 2020, 63/004,916 filed 3 Apr. 2020, 63/003,187 filed 31 Mar. 2020, 63/007,261 filed 8 Apr. 2020, 63/025,631 filed 15 May 2020, 63/025,837 filed 15 May 2020, and 63/142,392 filed 27 Jan. 2021, each of which is incorporated herein by reference as though fully set forth.

### SEQUENCE LISTING

The sequence listing contained in the electronic file titled “VAND-0208-PCT_sequence_listing_ST25.txt,” created 27 Mar. 2021 and comprising 131 kb, is hereby incorporated herein.

### BACKGROUND

### SARS-CoV-2

Severe acute respiratory syndrome coronavirus 2 (SARS-CoV-2 or sometimes 2019-nCoV), a spherical positive single-stranded RNA virus, has caused an ongoing pandemic of coronavirus disease 2019 (COVID-19). To date, SARS-CoV-2 has infected over 126 million individuals worldwide and resulted in over 2.7 million deaths. SARS-CoV-2 is a strain of the SARS-CoV species in the betacoronavirus genus.

As with other coronaviruses, SARS-CoV-2 has four structural proteins—the S (spike), E (envelope), M (membrane), and N (nucleocapsid). The S, E, and M proteins form the viral envelope, while the N protein, located in the core of the viral particle, binds the viral RNA and is responsible for its conformation into the viral particle. Some betacoronaviruses also include a hemagglutinin-esterase (HE) protein on the particle surface, which may enhance entry into the host cell.

The spike proteins are trimeric class I fusion proteins, heavily glycosylated, and project from the virion surface, promoting attachment to and entry into host cells. In some coronaviruses, the S protein consists of two subunits, S1 and S2, on the surface of the viral particle. In other coronaviruses, including SARS-CoV-2, the S protein includes S1 and S2 domains but remains intact on the viral particle surface until cleavage inside endocytic vesicles during viral entry.

Significant structural rearrangement of the S protein is involved in its fusion with a host cell membrane. Specifically, the receptor-binding domain (RBD) of the S1 subunit of the S protein undergoes a conformational movement from a “down” conformation, in which the binding receptors of the S1 subunit are inaccessible, to an “up” conformation, in which the receptors are accessible. The “up” conformation is believed to be less stable than the “down” conformation.

A precise three-dimensional structure of the S protein has recently been determined using cryogenic electron microscopy (cryo-EM). A representation of the three-dimensional S protein structure is shown in FIG. 7.

The S2 domain controls entry of the virus into the host cell. Angiotensin-converting enzyme 2 (ACE2), a type I membrane protein, is expressed widely across human tissues, including lung, heart, kidney, intestine, and adipose tissue. ACE2 has been identified as the host cell receptor for the earlier SARS-CoV strain and is a target of the SARS-CoV-2 S protein RBD.

RBD binding is believed to occur on the outer surfaces of the ACE2 protein, while angiotensin substrate binding occurs within a deep cleft of the protein. A representation of the three-dimensional structure of ACE2 is shown in FIG. 8.

Recently, the 3.5-angstrom-resolution structure of the S protein has been described. As noted above, the S protein is cleaved into two its S1 and S2 subunits. This cleavage of S proteins by host proteases is critical for viral infection and its exit from the cell via lysosomes.

During infection, the S protein is cleaved by host cell proteases, exposing a fusion peptide of the S2 domain. Cleavage of the S protein occurs between the S1 and S2 domains and subsequently within the S2 domain (S2′) proximal to the fusion peptide. This leads to the fusion of viral and cellular membranes and the release of the viral genome into the cytoplasm of the host cell. Cleavage at both sites is believed to be necessary for viral entry into a host cell.

The S1/S2 cleavage cite of SARS-CoV-2 is between the threonine and methionine at positions 696 and 697, respectively, of the S protein amino acid sequence, which is provided herein as SEQ ID 1. This S1/S2 cleavage site is identical to that of SARS-CoV and has been shown to be cleaved by cathepsin L (CatL or CTSL), a lysosomal cystein protease encoded by the CTSL1 gene. CatL is a C1 peptidase dimer comprising disulfide-linked heavy and light chains deriving from a protein precursor.

The cleavage site with the S2 domain of SARS-CoV-2, the S2′ site, is identical to that of SARS-CoV, located between the arginine and serine at positions 815 and 816 (SEQ ID 1), respectively. It is believed that this site, too, is cleaved by CatL or transmembrane protease, serine 2 (TMPRSS2) during viral entry. Inhibition of CatL or TMPRSS2 has been shown to suppress SARS-CoV infection.

SARS-CoV-2 also has a furin-like protease cleavage site not found in SARS-CoV, between the arginine and serine at positions 685 and 686 (SEQ ID 1), respectively. This site may be cleaved by furin during viral egress. The S protein of SARS-CoV-2 might be also primed by TMPRSS2. Inhibition of TMPRSS2 has been shown to suppress SARS-CoV infection. Furthermore, TMPRSS2 expression correlates with SARS-CoV infection in the upper lobe of the lung.

The function of TMPRSS2 itself is unknown. It is known to be highly expressed in the prostate. TMPRSS2 has also been found to be upregulated by androgenic hormones in prostate cancer cells. In addition, 50% of prostate cancers contain genomic rearrangements that lead to a juxtaposition of the androgen inducible promoter of the TMPRSS2 gene near the E26 transformation-specific (ETS) oncogenes, placing the ETS oncogenes under androgen control. TMPRSS2 has therefore been investigated for its potential role in the propagation and clinical course of prostate cancer.

Similar to SARS-CoV, SARS-CoV-2 enters the cell by the means of binding of cellular receptor(s) including ACE2. Decreased expression of ACE2 is associated with cardiovascular diseases.

The structural basis for this recognition has been recently mapped out and the cryo-EM structure of the full-length viral spike protein that targets human ACE2 complex has been reported. The SARS-CoV-2 S protein binds ACE2 at least 10 times more tightly than that of SARS-CoV and mediates receptor recognition.

Despite similarities between the SARS-CoV and SARS-CoV-2 S1/S2 and S2′ cleavage sites, it is unclear whether treatment strategies applicable to SARS-CoV would be similarly applicable to SARS-CoV-2. One study showed that several SARS-CoV RBD-specific monoclonal antibodies did not bind to the SARS-CoV-2 S protein. Another study, however, showed that CR3022, a SARS-CoV-specific monoclonal antibody, bound to the SARS-CoV-2 RBD with high affinity.

The genomes of SARS-CoV-2 samples isolated from patients of the current pandemic were shown to differ only slightly, perhaps by less than 0.2%, suggesting recent emergence in humans and quick detection of the virus since emergence. Thus, timely identification of effective strategies for treating SARS-CoV-2, before additional mutations arise as the virus spreads among human populations, would improve the overall effectiveness in responding to the ongoing pandemic.

Infections with this virus have resulted in a Mar. 11, 2020 declaration by the World Health Organization of a coronavirus disease 2019 (COVID-19) pandemic. A potentially fatal consequence of a SARS-CoV-2 infection in humans is a form of acute respiratory distress syndrome or ARDS.

### Severe COVID-19 Infection/ARDS

Common COVID-19 symptoms include cough, fever, fatigue, myalgias, and diarrhea. Severe COVID-19 symptoms typically begin one week after initial symptoms and include dyspnea and hypoxemia, with respiratory failure progressing in patients exhibiting these severe COVID-19 symptoms in the form of acute respiratory distress syndrome (ARDS). Severe COVID-19 infection may lead to acute injury to cardiac, kidney, and liver tissues, including failure of these organ systems. Once it develops, ARDS is often fatal.

In the early months of the pandemic, it became apparent that advanced age and comorbidities are associated with a higher risk of severe COVID-19 infection. Yet none of these fully explained the heterogeneous course of the infection observed.

More recently, genetic sequencing studies of COVID-19 patients suggest that a locus at 3p21.31 is associated with the severity of COVID-19 infection. The genetic variants on chromosome 3 that are most associated with severe COVID-19 are all in high linkage disequilibrium (LD). And recent phylogenetic analysis has showed that this haplotype of six genes (SLC6A20, LZTFL1, CCR9, FYCO1, CXCR6, and XCR1) for risk of severe COVID-19 infection entered the modern human genome from Neanderthals.

### Host Proteases

The host protease dependence of SARS-CoV-2 entry is critical to its infection efficacy. SARS-CoV takes advantage of the endosomal cysteine proteases cathepsin B (CTSB) and cathepsin L (CTSL). CTSL is a peptidase that preferentially cleaves peptide bonds with aromatic residues in P2 and hydrophobic residues in P3 position. CTSL is active at pH 3-6.5 in the presence of thiol and its enzymatic stability is dependent on ionic strength. CTSL proteolysis was shown to be an important mechanism in processing viral glycoprotein before cell membrane fusion during prior Ebola and SARS-CoV outbreaks.

Genetic variation in the CTSL gene could affect the propagation capacity of SARS-CoV-2. For example, CTSL polymorphisms could affect an individual's susceptibility to SARS-CoV-2 where, for example, individuals with certain genetic variant have reduced expression of CTSL. Such individuals could be relatively better protected or have lower viral titers as compared to individuals with other CTSL variants. Additionally, elements of a host's MHCI- and CTSL-mediated immune responses might affect viral proliferation. There are susceptibility factors spanning from ethnicity background to age related groups, to comorbid conditions.

### High-Throughput Screening Assays (HTSAs)

Elshabrawy et al., “Identification of a Broad-Spectrum Antiviral Small Molecule against Sever Acute Respiratory Syndrome Coronavirus and Ebola, Hendra, and Nipah Viruses by Using a Novel High-Throughput Screening Assay,” Journal of Virology 88(8):4353-4365 (2014), describe a high-throughput screening assay (HTSA) for identifying small molecules potentially useful in treating SARS-CoV, Ebola virus (EBOV), Hendra virus (HeV), and Nipah virus (NiV), all highly infectious zoonotic viruses. All of these viruses are encapsulated and require host proteases for their glycoprotein processing, cleavage, and entry into host cells.

The HTSA Elshabrawy et al. describe was useful in identifying several compounds capable of inhibiting pseudotyped viral entry of host cells by selectively inhibiting CatL cleavage of viral fusion peptides. The preferred compounds resulted in strong inhibition of such entry and significantly weaker inhibition of host peptide cleavage (e.g., pro-NYP-derived peptide).

### Human Leukocyte Elastase, D-Dimers and Alpha 1 Antitrypsin

D-Dimers are a breakdown product of a blood clot once the clot is broken down by fibrinolysis, also referred to as fibrin degradation products (FDP). D-Dimers are seen in inflammatory conditions and are believed to be a reflection of the activity of plasmin and elastase, two key proteases. The human leukocyte elastase (HLE) participates in fibrinolysis and its activity produces D-Dimers and also cleaves a number of other proteins, including elastin. Leukocyte elastase is derived from granulocytes which are often elevated during inflammation and are part of the host immune response. Increased activity of elastase in the lung is associated with emphysema. A prevailing hypothesis in the pathogenesis of Chronic Obstructive Pulmonary Disease (COPD) is the disturbance of the balance of proteases and their inhibitors, and specifically HLE and its natural inhibitor alpha 1 antitrypsin (A1AT). Moreover, extensive genetic evidence has shown that individuals with hereditary alpha 1 antitrypsin deficiency develop emphysema in adulthood.

### Coenzyme Q10

Oxidative stress plays a damaging role in viral infection, through multiple pathways, including diminishing the antioxidant response. The global scientific community is rapidly trying to delineate the pathophysiology of disease with SARS-CoV2 infection, associated biomarkers of severe illness, and potential therapeutics. An examination of associations between levels of critical antioxidants such as Coenzyme Q10 (CoQ10) and severity of SARS-CoV2 infection should be examined as potential associations may indicate markers of disease severity and possibly have a causative role.

CoQ10 is a fat-soluble molecule that is a member of the ubiquinone family (FIG. 13). CoQ10 is ubiquitous in humans and present in most cells and is both synthesized endogenously and acquired exogenously. The highest levels of CoQ10 are in the organs with the highest metabolic demand such as the heart, lungs, kidney and liver. CoQ10 has several important physiological roles including acting as an essential cofactor in the electron-transport chain to generate ATP and serving as a lipid antioxidant neutralizing free radicals and ensuing damage to the body.

Levels of CoQ10 can be diminished for several reasons including advanced age, exogenous compounds interfering with synthesis, and genetic conditions predisposing to lower levels. Statins inhibit HMG-CoA reductase, reducing synthesis of cholesterol and levels of CoQ10 due to the inhibition of a common pathway of synthesis. Atorvastatin was found to decrease the level of CoQ10 by 49% within 14 days of treatment. There is a peak of CoQ10 levels around age 20, followed by an age-dependent decrease over time. The largest tissue specific decrease at age 80 occur in the lungs (51.7% from peak) and heart (42.9% from peak). Mutations of several genes involved in CoQ10 biosynthesis can result in a deficiency.

CoQ10 has an integral anti-inflammatory role in the body as a free radical scavenger and has been explored extensively in the treatment of a variety of inflammatory mediated diseases. CoQ10 supplementation improved survival and pulmonary edema in sepsis-induced acute lung injury in rats. In patients with septic shock, CoQ10 levels were found to be lower and correlated with higher levels of inflammatory markers. Inhibition of platelet aggregation by CoQ10 may occur through multiple pathways including the upregulation of cAMP and PKA, and through the inhibition of vitronectin (CD51/CD61). CoQ10 has been found to be beneficial in attenuating fibrosis in the lung and liver in rats through up regulation of autophagy processes. Supplementation with CoQ10 improves liver and systemic markers of inflammation in people with nonalcoholic fatty liver disease. CoQ10 supplementation improves mortality and cardiac markers in people with heart failure. Total cholesterol and low-density lipoprotein levels improve in people with diabetes with CoQ10 supplementation. Supplementation with CoQ10 has been found to improve endothelial dysfunction in people with dyslipidemia.

Regarding the role in viral infection, CoQ10 has been shown to be lower in patients with acute influenza. A study of sixty-five children with influenza demonstrated that children with H1N1 had significantly lower levels of CoQ10 compared to the group with season influenza.

Although CoQ10 levels decrease over time and also through the consumption of exogenous agents such as statins, it has been seen that several genetic diseases also result in CoQ10 deficiencies. People with Down syndrome were found to have lower levels of CoQ10, and higher levels of TNF-alpha and IL-6. Further, people with Down syndrome have a higher susceptibility to viral and bacterial infections, a higher incidence of autoimmune diseases (diabetes, hypothyroidism), and a higher incidence of acute lung injury. Acute respiratory distress syndrome (ARDS) in people with Down syndrome has been postulated to be due to an imbalance in free radical scavengers.

### SUMMARY

One aspect of the invention includes a method of identifying compounds useful in treating or preventing infection by severe acute respiratory syndrome coronavirus 2 (SARS-CoV-2), the method comprising: screening at least one candidate compound for an ability to inhibit cleavage of a SARS-CoV-2 spike protein by a human protease at one or more target site.

Another aspect of the invention provides a method of predicting the efficacy of inhibiting enzymatic cleavage of severe acute respiratory syndrome coronavirus 2 (SARS-CoV-2) spike protein with a compound determined to have such inhibiting activity, the method comprising: determining whether the spike protein amino acid sequence includes one or more mutation from a wildtype sequence (SEQ ID 1), the one or more mutation being selected from a group consisting of: histidine at position 675 (SEQ ID 3), leucine at position 704 (SEQ ID 4), alanine at position 718 (SEQ ID 5), phenylalanine at position 752 (SEQ ID 6), leucine at position 765 (SEQ ID 7), leucine at position 772 (SEQ ID 8), glutamine at position 780 (SEQ ID 9), cysteine at position 797 (SEQ ID 10), and serine at position 812 (SEQ ID 11); and in the case that the spike protein amino acid sequence includes one or more such mutation, predicting that the compound will be effective in inhibiting enzymatic cleavage of the SARS-CoV-2 spike protein.

Still another aspect of the invention provides a method of inhibiting enzymatic cleavage of severe acute respiratory syndrome coronavirus 2 (SARS-CoV-2) spike protein, the method comprising: administering to an individual infected with SARS-CoV-2 or at risk of infection by SARS-CoV-2 a compound capable of binding to an octameric, nonameric, or decameric sequence of SEQ ID 2:

(SEQ ID 2)
GSFCTQLNRALTGIAVEQDKNTQ.

Another aspect of the invention provides a method of treating or preventing infection by severe acute respiratory syndrome coronavirus 2 (SARS-CoV-2) in an individual, the method comprising: administering to the individual amantadine in an amount sufficient to reduce lysosomal enzymatic activity in a cell of the individual.

Another aspect of the invention provides a method of decreasing a SARS-CoV-2 viral load in an individual infected therewith, the method comprising: administering to the individual amantadine in an amount sufficient to reduce lysosomal enzymatic activity in a cell of the individual.

Another aspect of the invention provides a method of predicting an individual's susceptibility to infection by SARS-CoV-2, the method comprising: determining a genotype of the individual at the rs2378757 locus; and in the case that the individual's rs2378757 genotype is AC or CC, predicting that the individual is relatively less susceptible to infection by SARS-CoV-2; or in the case that the individual's rs2378757 genotype is AA, predicting that the individual is relatively more susceptible to infection by SARS-CoV-2.

Yet another aspect of the invention provides a method of predicting the efficacy of treating an individual infected by SARS-CoV-2 with amantadine, the method comprising: determining a genotype of the individual at the rs2378757 locus; and in the case that the individual's rs2378757 genotype is AC or CC, predicting that treatment of the individual with amantadine will be relatively more effective; or in the case that the individual's rs2378757 genotype is AA, predicting that treatment of the individual with amantadine will be relatively less effective.

Still yet another aspect of the invention provides a method of reducing a risk of infection by SARS-CoV-2 in an individual, the method comprising: administering to the individual amantadine in an amount sufficient to reduce lysosomal enzymatic activity in a cell of the individual.

Another aspect of the invention provides a method of treating an individual infected with SARS-CoV-2 and suffering from or at risk for acute respiratory distress syndrome (ARDS), the method comprising: administering to the individual at least one human leukocyte elastase (HLE) inhibitor in an amount sufficient to decrease HLE activity in the individual.

Another aspect of the invention provides a method of treating an individual infected with SARS-CoV-2 and suffering from or at risk for acute respiratory distress syndrome (ARDS), the method comprising: administering to the individual at least one alpha 1 antitrypsin (A1AT) inducer or A1AT replacement in an amount sufficient to increase A1AT activity in the individual.

Another aspect of the invention provides a method of predicting a susceptibility to acute respiratory distress syndrome (ARDS) in an individual infected with SARS-CoV-2, the method comprising: determining, or having determined, an alpha 1 antitrypsin (A1AT) genotype of the individual; and in the case that the individual's A1AT genotype includes an S allele, a Z allele, or both, predicting that the individual has an increased susceptibility to ARDS; or in the case that the individual's A1AT genotype includes neither an S allele nor a Z allele, predicting that the individual does not have an increased susceptibility to ARDS.

Another aspect of the invention provides a method of treating patients diagnosed with or, based on symptoms and possible exposure, are suspected to be infected with a coronavirus 2 (SARS-CoV-2) infection that has resulted in acute respiratory distress syndrome (ARDS). The method involves administering to such an individual an amount of an antiandrogenic agent that is effective to reduce transmembrane protease, serine 2 (TMPRSS2) activity in the individual to a level sufficient to ameliorate the manifestations (i.e., one or more symptoms or other physiological effects) of ARDS. A related aspect of the invention provides a method of reducing transmembrane protease, serine 2 (TMPRSS2) activity in an individual by administering to an individual at least one antiandrogenic agent.

Most specifically, the method includes treating an individual who is diagnosed as having, or is susceptible to development of, severe acute respiratory distress syndrome as a consequence of a diagnosed or suspected coronavirus 2 (SARS-CoV-2) infection, comprising administering to the individual an antiandrogenic agent in an amount effective to reduce transmembrane protease, serine 2 (TMPRSS2) activity in the individual to a level sufficient to ameliorate the manifestations of ARDS.

In a further embodiment of the present invention, a method of is provided for reducing transmembrane protease, serine 2 (TMPRSS2) activity in an individual by administering to the individual at least one antiandrogenic agent.

For the purposes of the present above methods, the antiandrogenic agent is one or more an androgen receptor (AR) antagonist, androgen synthesis inhibitors, or antigonadotropins. In this regard, in one embodiment, the antiandrogenic agent is one or more AR antagonists selected from a group consisting of: cyproterone acetate, megestrol acetate, chlormadinone acetate, spironolactone, oxendolone, osaterone acetate, dienogest, drospirenone, medrogestone, nomegestrol acetate, promegestone trimegestone, flutamide, bicalutamide, nilutamide, topilutamide, enzalutamide, and apalutamide. In another embodiment, the antiandrogenic agent is one or more androgen synthesis inhibitors selected from a group consisting of ketoconazole, abiraterone acetate, seviteronel, aminoglutethimide, finasteride, dutasteride, epristeride, alfatradiol, and Serenoa repens extract. In yet another embodiment, the antiandrogenic agent is one or more antigonadotropins selected from a group consisting of cetrorelix, allylestrenol, chlormadinone acetate, cyproterone acetate, gestonorone caproate, hydroxyprogesterone caproate, medroxyprogesterone acetate, megestrol acetate, osaterone acetate, oxendolone, estradiol, estradiol esters, ethinylestradiol, conjugated estrogens, and diethylstilbestrol.

In another aspect, the invention provides a method of identifying that a compound is a potentially useful candidate for treating infection of an individual or preventing infection of an individual or a cell by severe acute respiratory syndrome coronavirus 2 (SARS-CoV-2) comprising: determining that the compound binds to angiotensin-converting enzyme 2 (ACE2) such that subsequent binding by the SARS-CoV-2 spike protein is blocked at one or more amino acid selected from a group consisting of: histidine at position 34 of SEQ ID 1, aspartate at position 30 of SEQ ID 1, tyrosine at position 41 of SEQ ID 1, glutamine at position 42 of SEQ ID 1, lysine at position 353 of SEQ ID 1, and arginine at position 453 of SEQ ID 1; or determining that the compound binds to the SARS-CoV-2 spike protein such that subsequent binding is blocked at one or more amino acids selected from a group consisting of:

tyrosine at position 453 of SEQ ID 2, glutamine at position 498 of SEQ ID 2, threonine at position 500 of SEQ ID 2, asparagine at position 501 of SEQ ID 2, and lysine at position 417 of SEQ ID 2. Such determining may include calculating one or more terms selected from a group consisting of: a Van der Waals energy term, a Coulomb energy term, a lipophilic term, a hydrogen-bonding term, a metal-binding term, a reward term, and a penalty term.

In another aspect, the invention provides a method of inhibiting sudden acute respiratory syndrome coronavirus 2 (SARS-CoV-2) infection of a cell manifesting angiotensin-converting enzyme 2 (ACE2) on the cell membrane, the method comprising: exposing the cell to a concentration of a compound capable of preventing binding of the SARS-CoV-2 spike protein to the ACE2 on the cell.

In another aspect, the invention provides a method of treating an individual infected with severe acute respiratory syndrome coronavirus 2 (SARS-CoV-2), the method comprising: administering to the individual an amount of a compound effective to prevent binding of the SARS-CoV-2 spike protein to angiotensin-converting enzyme 2 (ACE2) on cells of the individual exhibiting ACE2 on the cell membranes.

In still another aspect, the invention provides a method of preventing infection of an individual with severe acute respiratory syndrome coronavirus 2 (SARS-CoV-2) who is at risk of such infection, the method comprising: administering to the individual an amount of a compound effective to prevent binding of the SARS-CoV-2 spike protein to angiotensin-converting enzyme 2 (ACE2) on the cells of the individual exhibiting ACE2 on the cell membranes.

Another aspect of the invention provides a method of treating or preventing severe acute respiratory syndrome coronavirus 2 (SARS-CoV-2) infection in an individual, the method comprising: determining that a level of coenzyme Q10 of the individual is lower than expected; and administering to the individual a quantity of coenzyme Q10.

Still another aspect of the invention includes a method of treating a patient suffering from a betacoronavirus infection, the method comprising: determining, or having determined, whether the patient carries at least one genetic marker associated with severe betacoronavirus infection; and in the case that the patient carries the at least one genetic marker associated with severe betacoronavirus infection, administering to the patient an effective amount of at least one compound capable of inhibiting expression of the FYCO1 gene, the at least one compound being selected from a group consisting of: indomethacin, primidone, triprolidine hydrochloride, and baclofen.

Another aspect of the invention provides a method of determining a predisposition to severe betacornonavirus infection in an individual, the method comprising: determining, or having determined, whether the individual carries at least one marker associated with severe betacoronavirus infection; and in the case that the individual carries the at least one marker associated with severe betacoronavirus infection, determining that the individual is predisposed to severe betacoronavirus infection.

### BRIEF DESCRIPTION OF THE DRAWINGS

These and other features of the invention will be more readily understood from the following detailed description of the various aspects of the invention taken in conjunction with the accompanying drawings that depict various embodiments of the invention, in which:

FIG. 1 shows the effect of amantadine on lysosome pathway genes.

FIG. 2 shows expression of CTSL across different organs and associated cell types.

FIG. 3 shows the relative expression of rs2378757 variants in lung tissue.

FIG. 4 shows alternative splicing of the CTSL transcript in various tissue types.

FIG. 5 shows the three-dimensional structure of the human neutrophil elastase protein in panel (a) and the three-dimensional structure of alpha 1 antitrypsin in panel (b).

FIG. 6 shows a heat map of Pi*SZ in Europe, as reported in one study.

FIG. 7 is a representation of the three-dimensional structure of the SARS-CoV-2 S protein.

FIG. 8 is a representation of the three-dimensional structure of ACE2.

FIGS. 9A and 9B are, respectively, a graphical representation of the binding affinity analyses of the AY-NH₂ compound for ACE2 and a representation of the three-dimensional structure of the AY-NH₂ compound bound to ACES.

FIGS. 10A and 10B are, respectively, a graphical representation of the binding affinity analyses of the NAD⁺ compound for ACE2 and a representation of the three-dimensional structure of the NAD⁺ compound bound to ACES.

FIGS. 11A and 11B are, respectively, a graphical representation of the binding affinity analyses of the reproterol compound for ACE2 and a representation of the three-dimensional structure of the reproterol compound bound to ACES.

FIGS. 12A and 12B are, respectively, a graphical representation of the binding affinity analyses of the thymopentin compound for ACE2 and a representation of the three-dimensional structure of the thymopentin compound bound to ACES.

FIG. 13 shows the molecular structure of Coenzyme Q10.

It is noted that the drawings are not to scale and are intended to depict only typical aspects of the invention, and therefore should not be considered as limiting the scope of the invention.

### DETAILED DESCRIPTION

### Screening Assay

Applicant has identified a number of novel cleavage sites for CatL in the SARS-CoV-2 S protein sequence that may be relevant in the treatment or prevention of SARS-CoV-2 infection. Any compound, including small molecules, having an ability to inhibit cleavage at one or more of these sites—whether by direct inhibition of enzymatic cleavage by binding to a host protease, binding to the S protein to block these cleavage sites, altering expression of the host protease, or altering a function of a lysosome in which the host protease is contained—may be useful in treating or preventing SARS-CoV-2 infection.

The novel cleavage sites identified by Applicant comprise locations of the wildtype amino acid sequence of the S protein (SEQ ID 1) at which protease cleavage is known or believed to occur, whether during viral entry, replication, packaging, or egress. One skilled in the art will recognize that other amino acid sequences for the SARS-CoV-2 S protein, representing minor deviations from the wildtype sequence, are known. The novel cleavage sites of the invention are found in these alternative sequences as well, although the particular amino acid positions may differ.

The novel cleavage sites of the invention include those located between the threonine and glycine at positions 768 and 769 (SEQ ID 1), respectively, and the arginine and serine at positions 815 and 816 (SEQ ID 1), respectively.

Embodiments of the invention include methods of screening one or more candidate compound for an ability to inhibit cleavage of the SARS-CoV-2 S protein at either of these novel cleavage sites. Such screening includes simultaneously or sequentially screening a plurality of candidate compounds using an assay.

Assays useful in practicing the invention include, for example, the HTSA described by Elshabrawy et al., which is hereby incorporated by reference for all that it teaches, as though fully set forth.

Other embodiments of the invention include inhibiting enzymatic cleavage of the SARS-CoV-2 spike protein, treating SARS-CoV-2 infection, or preventing SARS-CoV-2 infection by administering to an individual at least one compound identified as having such an inhibitory ability.

In fact, Applicant has discovered a sequence range within the S protein that appears susceptible both to enzymatic cleavage and potential binding to inhibit such cleaving. That sequence is 23 amino acids in length, extending from the glycine at position 757 (SEQ ID 1) to the glutamine at position 779 (SEQ ID 1). This sequence is shown in full below and identified specifically herein as SEQ ID 2.

(SEQ ID 2)
GSFCTQLNRALTGIAVEQDKNTQ

Any amino acid sequence within SEQ ID 2 may be targeted for binding. As will be understood by one skilled in the art, such sequences should be of sufficient length to ensure proper binding specificity. Any octameric, nonameric, or decameric sequence within SEQ ID 2 would be expected to provide sufficient binding specificity.

Applicant has also discovered that one or more mutations in the SARS-CoV-2 S protein sequence may make inhibition of enzymatic cleavage more effective. It is believed that these mutations result in a conformational change in the S protein such that either of these novel cleavage sites is less susceptible to enzymatic cleavage. The presence of one or more of these mutations may also improve the inhibition of cleavage at the known CatL cleavage sites noted above, either by known compounds or those identified according to embodiments of the invention.

These mutations are shown below in Table 1. Mutations are described in terms of their mutation from an amino acid in the wildtype sequence (SEQ ID 1) at a particular position, as will be understood by one skilled in the art.

TABLE 1

| SEQ ID | name | mutation |
|---|---|---|
| 3 | Q675H | glutamine to histidine at position 675 |
| 4 | S704L | serine to leucine at position 704 |
| 5 | T719A | threonine to alanine at position 719 |
| 6 | L752F | leucine to phenylalanine at position 752 |
| 7 | R765L | arginine to leucine at position 765 |
| 8 | V772L | valine to leucine at position 772 |
| 9 | E780Q | glutamate to glutamine at position 780 |
| 10 | F797C | phenylalanine to cysteine at position 797 |
| 11 | P812S | proline to serine at position 812 |

Thus, embodiments of the invention may further include determining whether an individual is infected or at risk of being infected by a strain of the SARS-CoV-2 virus that includes one or more of the mutations in Table 1.

Of course, as should be apparent to one skilled in the art, determining whether the spike protein sequence includes one or more such mutations, and therefore is more susceptible to inhibition of enzymatic cleavage, is applicable to screening and treatment methods other than those described herein. Such a determining step may be used, for example, in treating an individual with a compound identified according to methods other than those described herein.

Understanding the mechanism of action of SARS-CoV-2 infection is a fundamental step in delineating the optimal therapeutic agents. For example, interfering with the S protein processing by the host cell, whether by affecting the environment or modulating gene expression levels, offers one potential therapeutic strategy.

Novel therapeutics identified by high throughput screening assay and shown to block the cleavage of SARS-CoV2-S protein by CTSL, CTSB, TMPRSS2, or any other host protease at predicted/selected binding sites will be a viable approach to functionally target and limit infection by SARS-CoV-2.

Other therapeutic mechanisms of action could involve lowering or modulating the expression of CTSL or affecting the conditions of the CTSL lysosomal environment by modulating pH.

Applicant has tested various agents that could help identify potential therapeutics with the capacity to decrease expression of the CTSL gene. Five such agents showed such potential, one of which is amantadine, and merit consideration as potentially useful in treating patients with COVID-19 infection.

### Cell Culture and Drug Treatment

A drug screen was used to test agents having the potential to treat or prevent SARS-CoV-2 infection. The retinal pigment epithelia cell line, ARPE-19/HPV-16, was chosen to establish a database of drug profiles because of its non-cancerous, human origin, with a normal karyotype. ARPE-19/HPV-16 can be easily grown as monolayer in 96-well plates and expresses a variety of well known, neuronal, cell surface receptors that include the dopamine receptor D2, the serotonin receptors 1A, 2A, and 2C, the muscarinic receptor M3, and the histamine receptor H1.

Cell lines were propagated according to supplier's specifications (ATCC Manassas, VA). Compounds were obtained from Sigma (St. Louis, MO) or Vanda Pharmaceuticals (Washington, DC). Cells were aliquoted on 96-well plates (˜2×10⁵ cells/well) and incubated for 24 hours prior to providing fresh media with a drug, or the drug vehicle (water, dimethyl sulfoxide, ethanol, methanol, or phosphate-buffered saline solution). Drugs were diluted 1000-fold in buffered Advanced D-MEM/F-12 culture medium (Invitrogen, Carlsbad, CA) containing nonessential amino acids and 110 mg/L sodium pyruvate. In these conditions, no significant changes of pH were expected, which was confirmed by the monitoring of the pH indicator present in the medium.

A final 10 μM drug concentration was chosen because it is believed to fit in the range of physiological relevance. Microscopic inspection of each well was conducted at the end of the treatment to discard any samples where cells had morphological changes consistent with apoptosis. It was also verified that the drug had not precipitated in the culture medium.

### Gene Expression Assay

Cells were harvested 24 hours after treatment and RNA was extracted using the RNeasy 96 protocol (Qiagen, Valencia, CA). Gene expression for 22,238 probe sets of 12,490 genes was generated with U133A2.0 microarrays following the manufacturer's instructions (Affymetrix, Santa Clara, CA). Drugs were profiled in duplicate or triplicate, with multiple vehicle controls on each plate. A total of 708 microarrays were analyzed including 74 for 18 antipsychotics, 499 for 448 other compounds, and 135 for vehicle controls.

The raw scan data were first converted to average difference values using MAS 5.0 (Affymetrix). The average difference values of both treatment and control data were set to a minimum threshold of 50 if below 50. For each treatment instance, all probe sets were then ranked based on their amplitude, or level of expression relative to the vehicle control (or average of controls when more than one was used). Amplitude was defined as the ratio of expression (t−v)/[(t+v)/2] where t corresponds to treatment instance and v to vehicle instance.

Each drug group profile was created using a novel Weighted Influence Model, Rank of Ranks (WIMRR) method which underscores the rank of each probe set across the entire gene expression profile rather than the specific change in expression level. WIMRR takes the average rank of each probe set across all of the members of the group and then re-ranks the probe sets from smallest average rank to largest average rank. A gene-set enrichment metric based on the Kolmogorov-Smirnov (KS) statistic. Specifically, for a given set of probes, the KS score gives a measure of how up (positive) or down (negative) the set of probes occurs within the profile of another treatment instance.

### Results

Applicant analyzed expression profile of CTSL across all 466 drugs tested. In order to find positive hits and selected only those results that caused decrease of CTSL expression by at least 33% (1.5-fold down). Top drug targets (Table1) included drugs from various therapeutic areas—muscle relaxer, antihistamine, anti-epileptic, anticholirgenic, and antiviral. There was no drug that would decrease CTSL expression by more than 40%. Among the top results is amantadine, a known and safe antiviral agent that was previously used to treat patients with influenza A.

TABLE 2
List of top drugs affecting CTSL downregulation

| | log2(treated) | log2(control) | log2 |
|---|---|---|---|
| DrugID | CTSL | CTSL | difference |
| Baclofen | 9.58 | 10.40 | −0.82 |
| Triprolidine Hydrochloride | 9.54 | 10.33 | −0.79 |
| Brompheniramine Maleate | 9.57 | 10.33 | −0.75 |
| Amantadine Hydrochloride | 9.62 | 10.33 | −0.70 |
| Phenytoin | 9.56 | 10.26 | −0.70 |
| Atropine Sulfate | 9.63 | 10.33 | −0.70 |

Amantadine hydrochloride is a lysosomotropic alkalinizing agent. The physical chemical properties of amantadine lead to lysosomal accumulation. Lysosomotropic drugs affect lysosomes by pH alteration, blocking Ca²⁺ signaling, lysosomal membrane permeabilizations, enzyme activity inhibition, and storage material accumulation. Amantadine behaves as a lysosomotropic substance that passes easily through the lysosome membrane and accumulates in the lysosome. It may lower the pH of the lysosome, thereby inhibiting protease activity.

Amantadine can also block the assembly of influenza virus during viral replication. Moreover, amantadine may directly affect viral entry by down-modulating CTSL and other lysosomal pathway genes.

Amantadine HCl IR is available as a 100-mg tablet (equivalent to 81 mg base amantadine) and 50 mg/S mL syrup (equivalent to 40 mg/S mL base amantadine) and is typically administered twice daily.

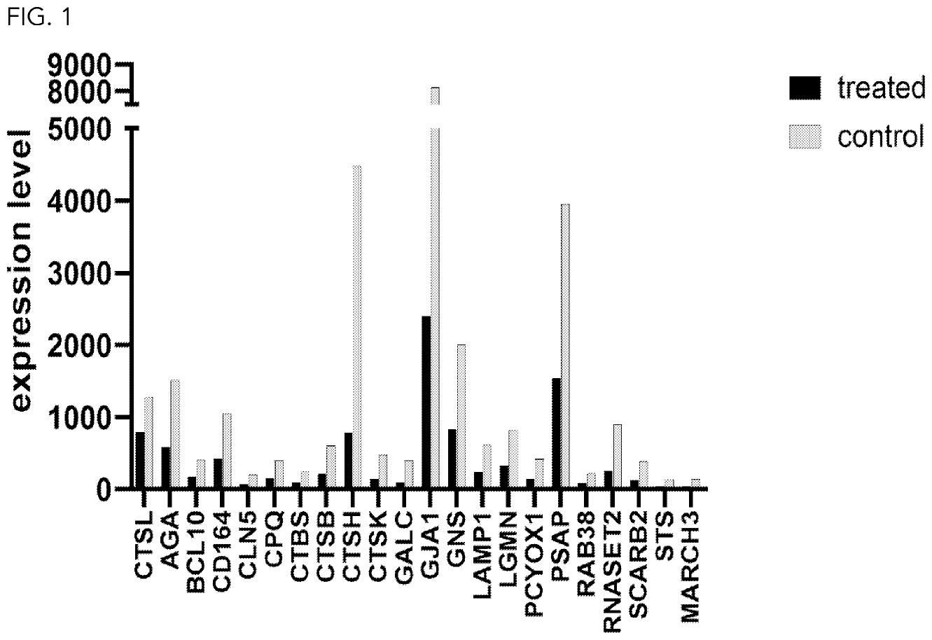

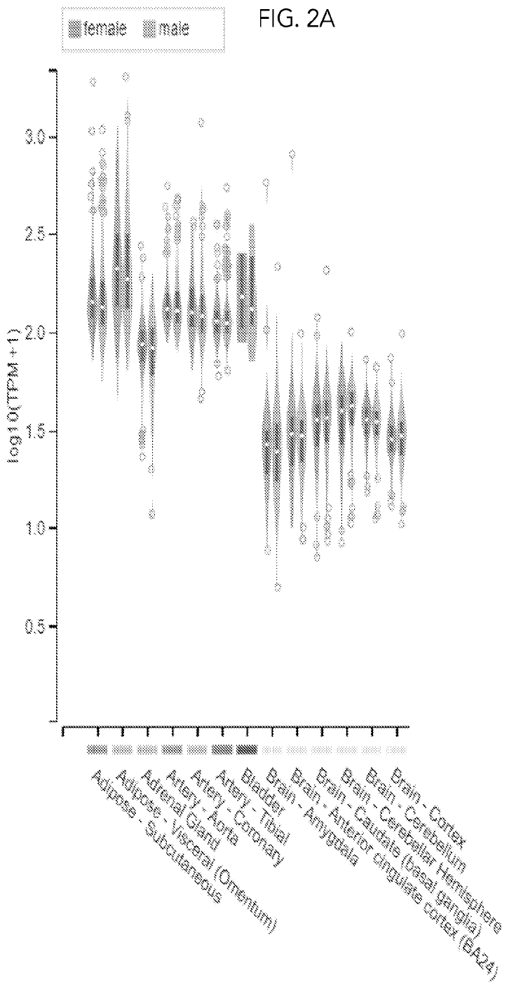

Since CTSL was not the top differentially expressed transcript, Applicant extended the analysis to all genes that were downregulated by amantadine. Among the top 500 differentially expressed probes (383 genes, all with at least 50% expression reduction) Applicant found 21 genes related to lysosomes (GO:005764, p=2.49×10⁻⁵). In addition, the top significant pathway by ENRICHR enrichment analysis is KEGG lysosome. Amantadine's significant effect on lysosome-associated membrane glycoprotein (LAMP) pathway genes is shown in FIG. 1 and Tables 3 and 4.

TABLE 3

| | | | % | Fold | | P-value | P-value |
|---|---|---|---|---|---|---|---|
| Term | Category | Count | Pathway | Enrichment | P-value | Bonferroni | FDR |
| GO:0005764-lysosome | GOTERM_CC_DIRECT | 21 | 5.19 | 4.42 | 6.65E−08 | 2.49E−05 | 9.39E−09 |
| Lysosome | UP_KEYWORDS | 19 | 4.69 | 3.75 | 3.77E−06 | 0.001389 | 0.005198 |
| hsa04142:Lysosome | KEGG_PATHWAY | 14 | 3.46 | 4.37 | 1.55E−05 | 0.003425 | 0.019698 |

TABLE 4

| | Probe | avg_tx | avg_ctrl | diff |
|---|---|---|---|---|
| Gene | ID | (log2) | (log2) | (log2) |
| CTSH | 202295_s_at | 9.62 | 12.13 | −2.51 |
| GALC | 204417_at | 6.58 | 8.64 | −2.06 |
| RNASET2 | 217983_s_at | 8 | 9.82 | −1.81 |
| CTSK | 202450_s_at | 7.09 | 8.9 | −1.81 |
| GJA1 | 201667_at | 11.23 | 12.99 | −1.76 |
| ceroid-lipofuscinosis | 204085_s_at | 5.93 | 7.67 | −1.74 |
| SCARB2 | 201647_s_at | 6.97 | 8.6 | −1.63 |
| AGA | 204333_s_at | 6.57 | 8.17 | −1.6 |
| ceroid-lipofuscinosis | 214252_s_at | 6.13 | 7.71 | −1.57 |
| MAR3 | 213256_at | 5.55 | 7.12 | −1.57 |
| PCYOX1 | 203803_at | 7.15 | 8.71 | −1.57 |
| CPQ | 203501_at | 6.41 | 7.96 | −1.55 |
| CTSB | 213274_s_at | 7.75 | 9.23 | −1.48 |
| steroid sulfatase | 203767_s_at | 5.55 | 7.02 | −1.47 |
| (microsomal) | | | | |
| LAMP1 | 201551_s_at | 7.87 | 9.27 | −1.4 |
| AGA | 204332_s_at | 9.18 | 10.57 | −1.39 |
| AGA | 216064_s_at | 8.12 | 9.5 | −1.38 |
| PSAP | 200866_s_at | 10.59 | 11.95 | −1.36 |
| LGMN | 201212_at | 8.33 | 9.68 | −1.35 |
| CPQ | 208454_s_at | 7.3 | 8.65 | −1.34 |
| CD164 | 208654_s_at | 8.7 | 10.04 | −1.34 |
| CTBS | 218924_s_at | 6.59 | 7.91 | −1.32 |
| RAB38 | 219412_at | 6.51 | 7.81 | −1.3 |
| N-acetyl-6-sulfatase | 212334_at | 9.71 | 10.97 | −1.27 |
| BCL10 | 205263_at | 7.44 | 8.69 | −1.25 |

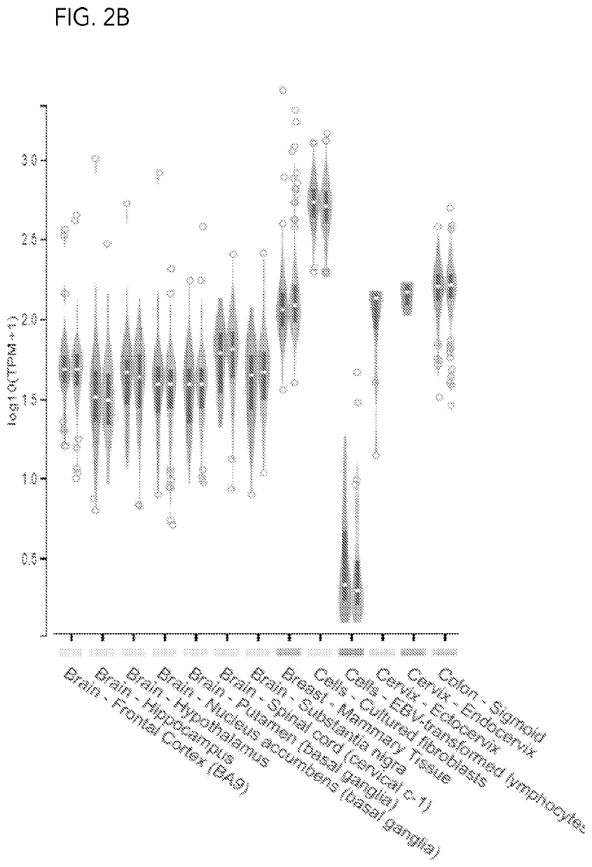

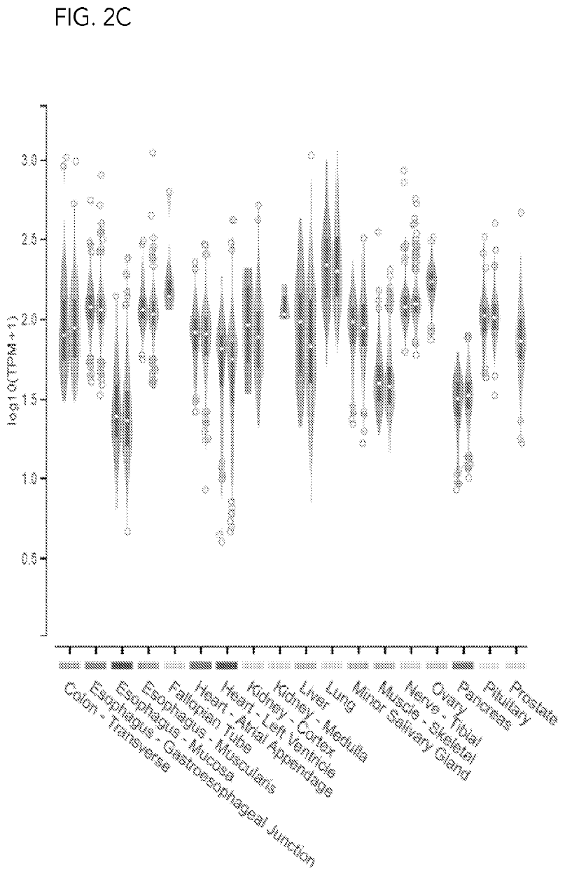

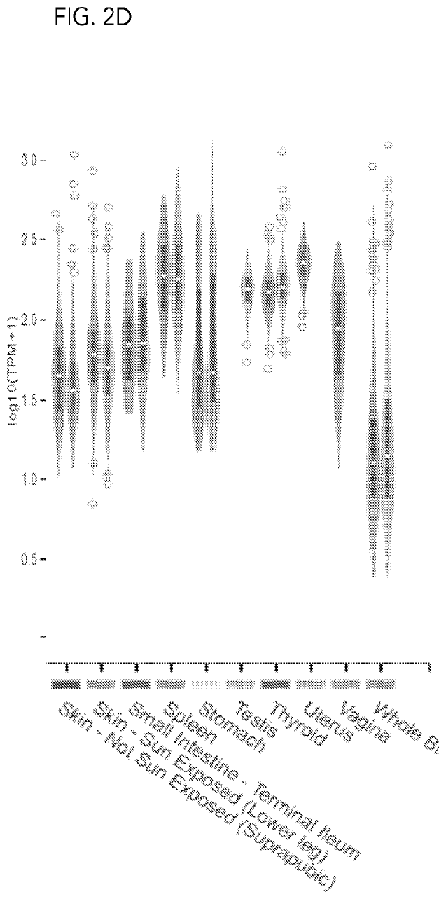

Applicant has also investigated the natural variation of CTSL expression across ethnicities, focusing on common and rare variants. The Genotype-Tissue Expression (GTEx) project provides genotype information and gene expression levels across 49 human tissues from 838 donors, allowing examination of the expression patterns of CTSL, both across tissues and across individuals. FIG. 2 shows the expression of CTSL across different organs and associated cell types. CTSL is widely expressed in many crucial organs (high in lungs, nerve tibial, adipose, artery, whole blood, etc.).

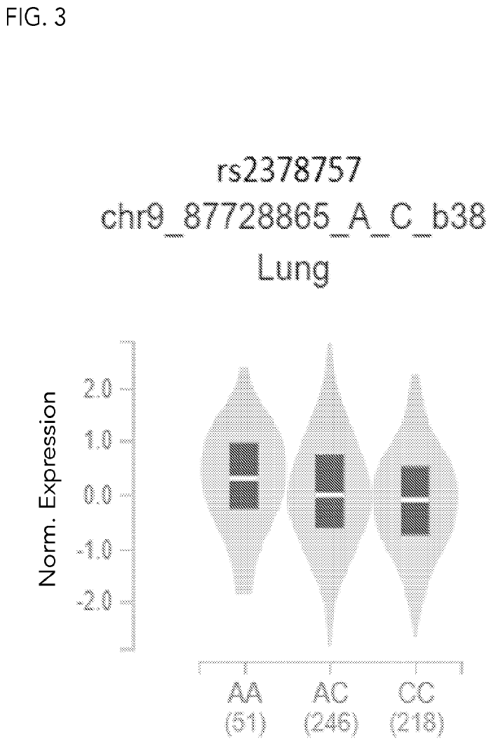

Looking at eQTL variants in CTSL, Applicant found a very significant and lung specific (rs2378757) variant conferring highly variable expression. As shown in FIG. 3, the CC genotype confers lower baseline expression and is likely associated with a better treatment response, while the AA genotype conversely confers a higher expression and perhaps a susceptibility to a higher viral load.

Applicant notes a series of splice QTLs, with variants affecting splicing ratios of transcripts, such as rs114063116, which was significant and present in lung tissue. CTSL GTEx analysis points to potential protection or susceptibility of certain individuals.

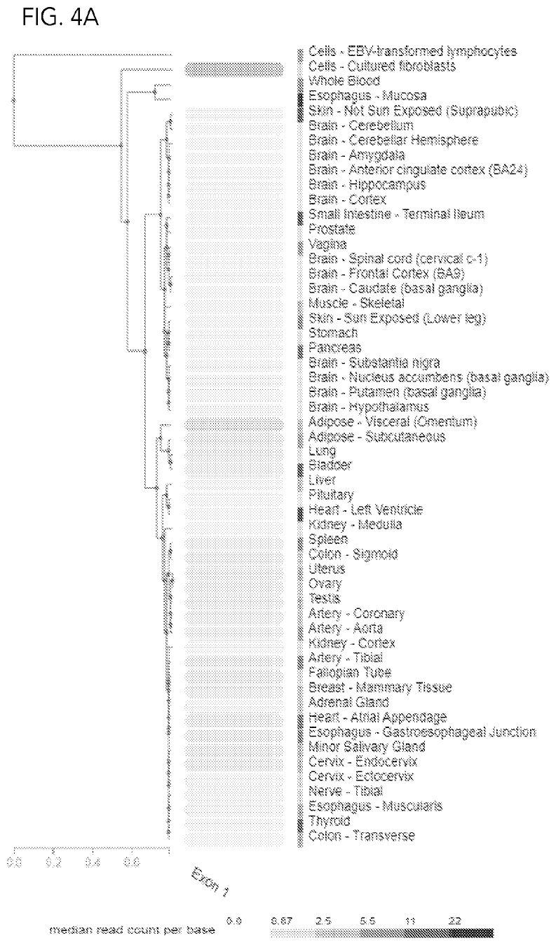

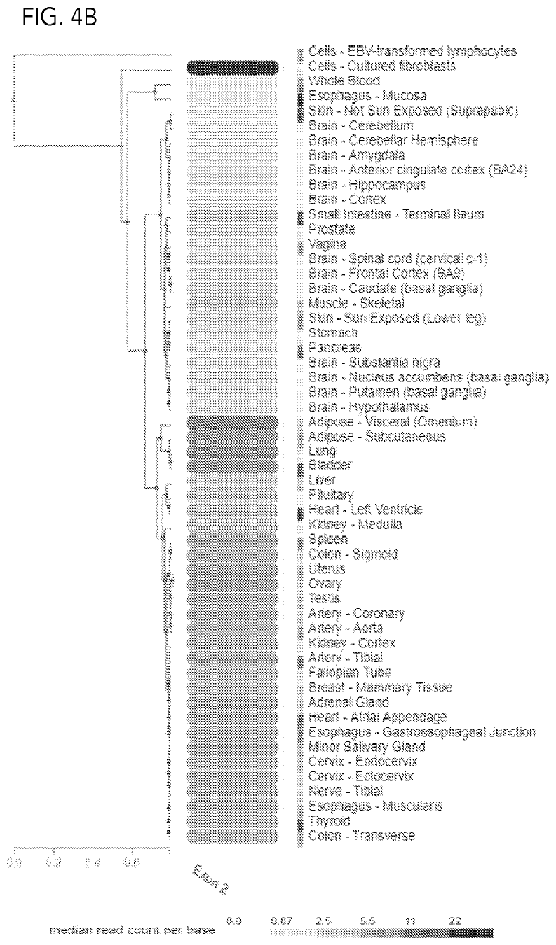

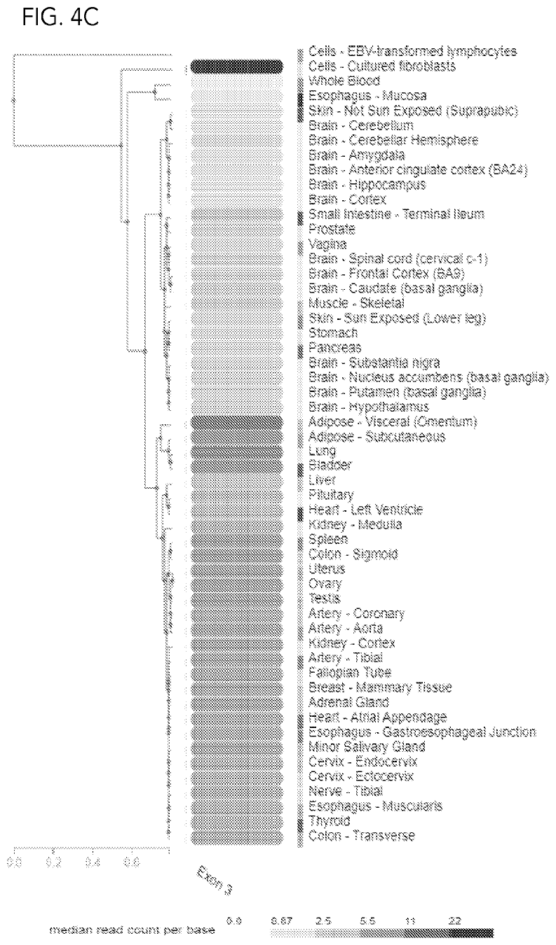

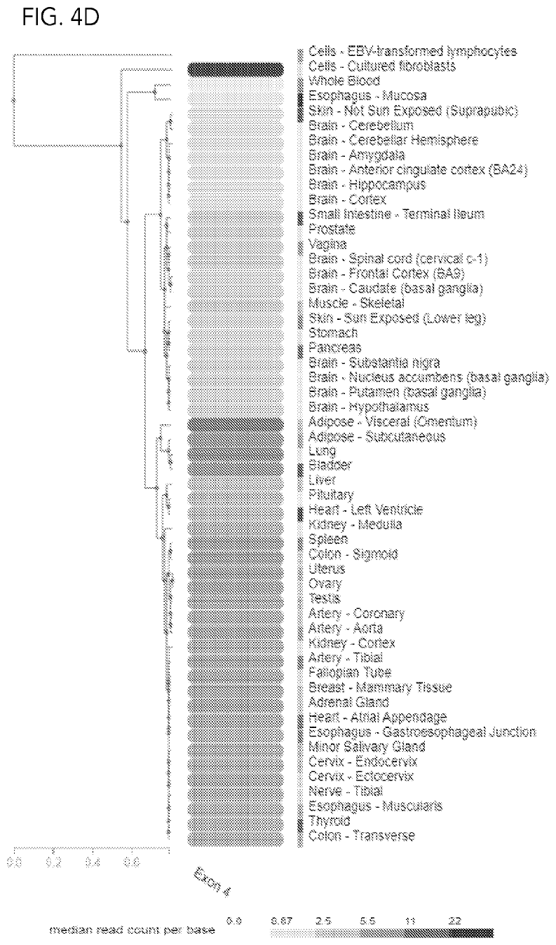

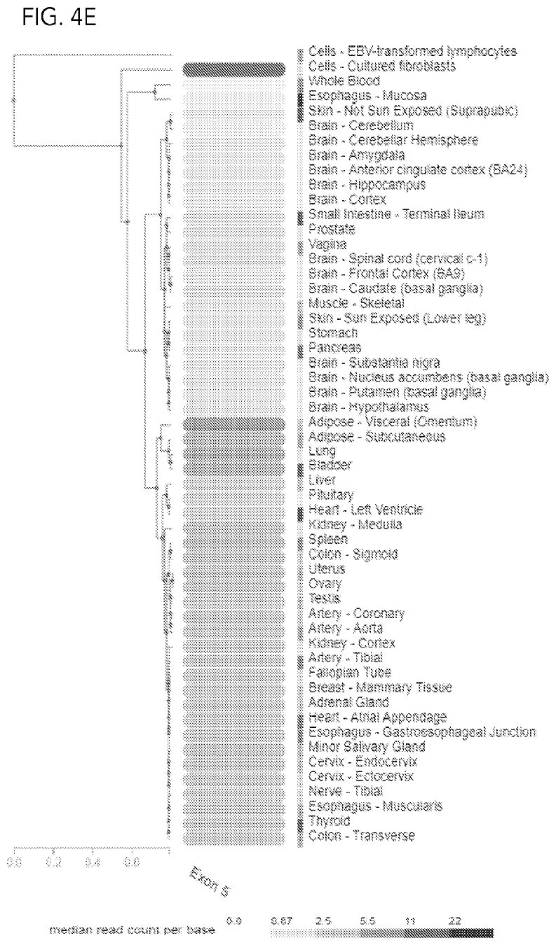

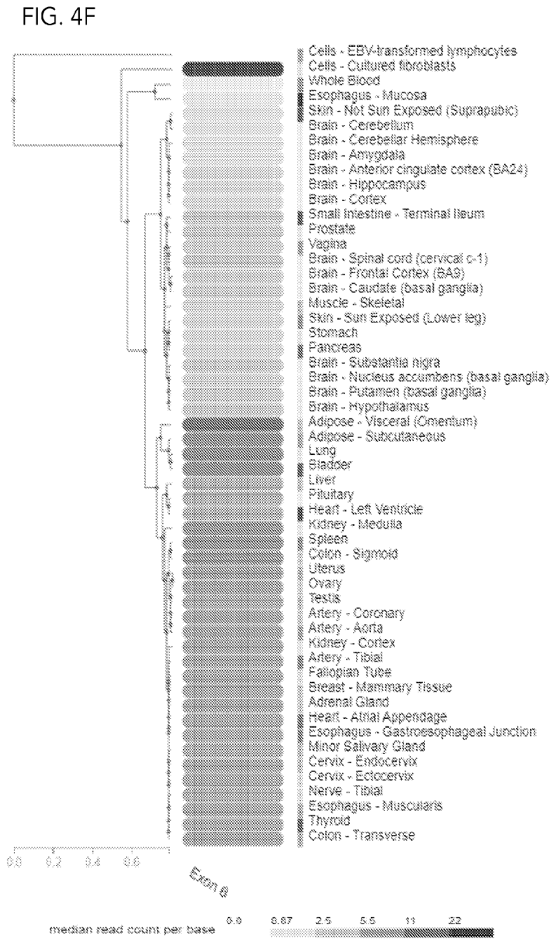

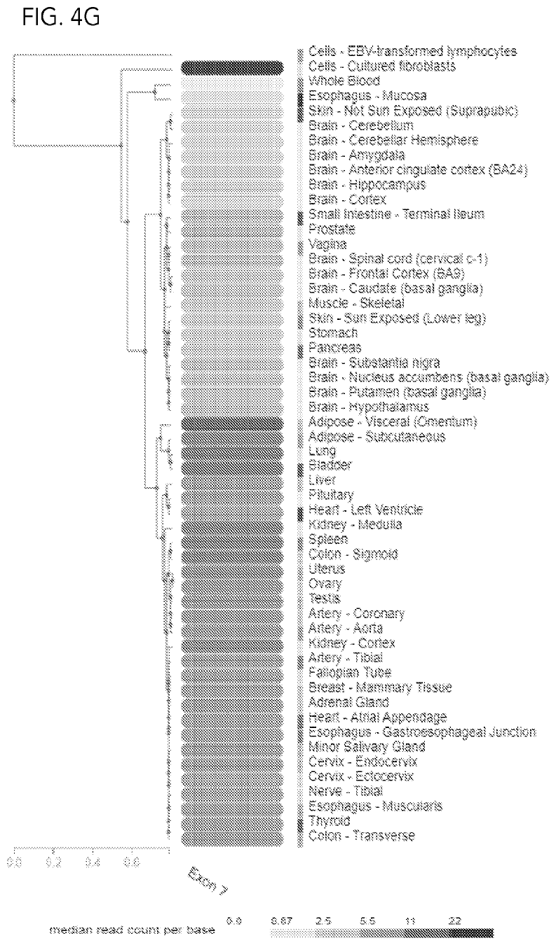

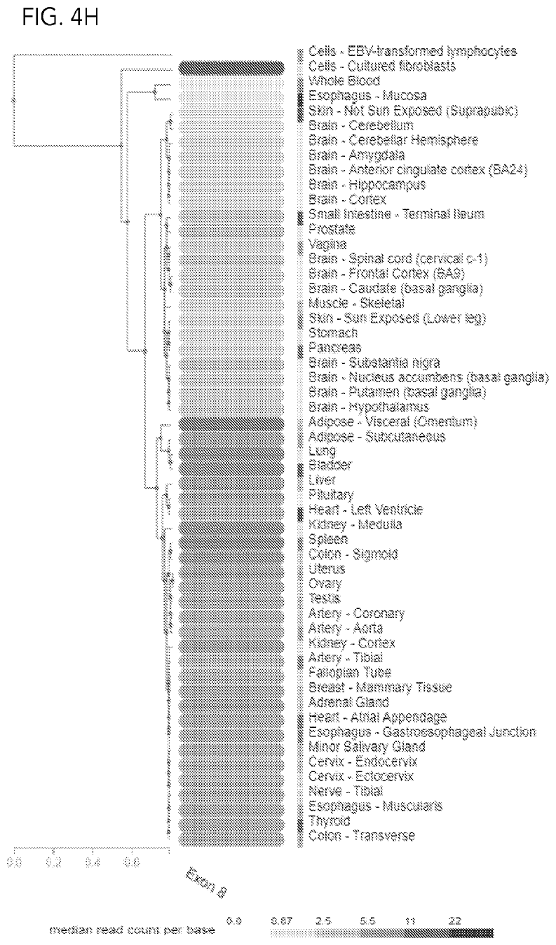

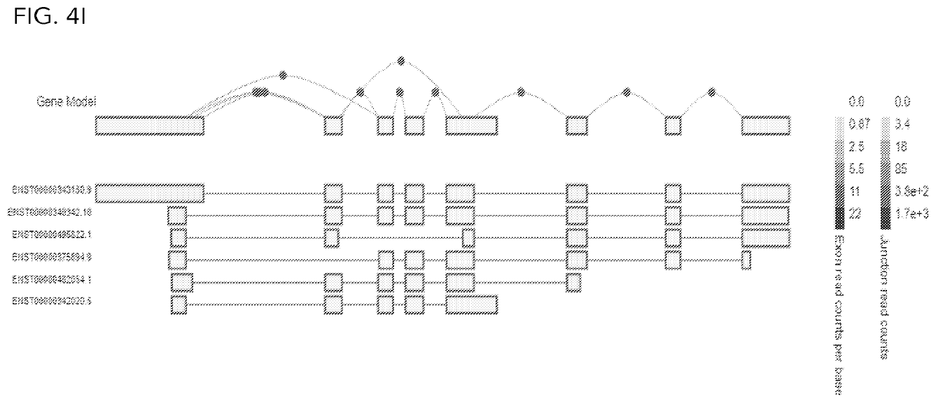

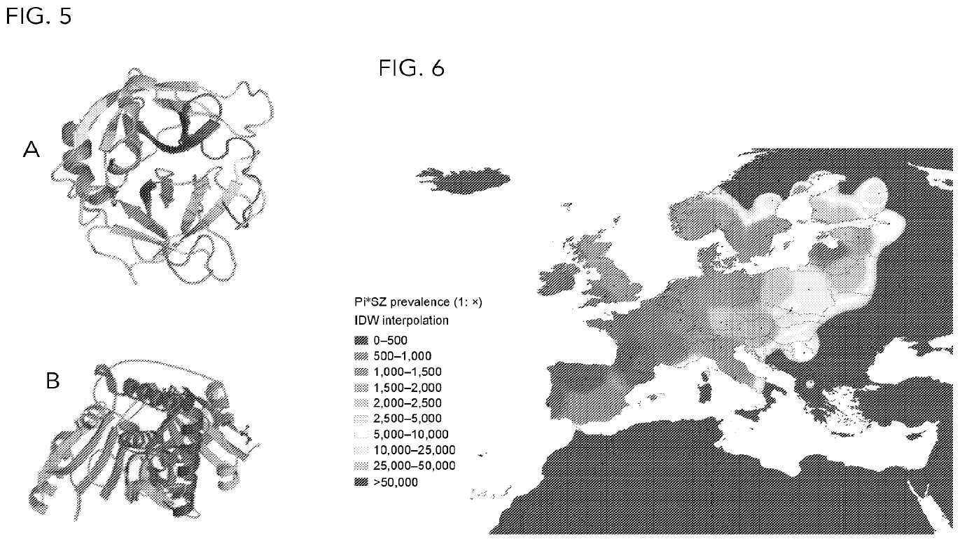

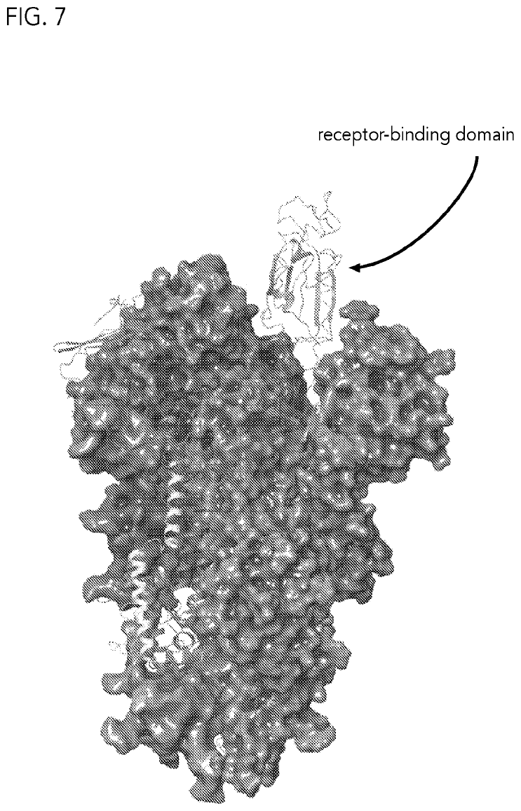

Interestingly, the alternative splicing of the CTSL transcript in the lung further displays tissue-specific regulatory programs. Results for various tissue types are shown in FIG. 4.

A recent functional study points to a common variant in CTSL in the proximal CTSL1 promoter (position C-171A) confirmed to alter transcription via alteration of the xenobiotic response element. This and similar other variants likely affect the natural diversity in baseline expression and therefore viral fitness at cell entry of SARS-CoV-2.

Additionally, Applicant notes, in the gnomAD database, a number of rare variants and variation tolerance statuses of CTSL. The results show that there are on average 167 missense variants in CTSL and the gene is predicted to be loss-of-function variant tolerant with a pLI of 0.01. Together with significant eQTLs, this indicates a large effect of genetic variation on CTSL expression and variation thereof.

TMPRSS2 is also widely expressed in multiple tissues, including those in the GI system, lung, and kidney. The high expression of CTSL and TRMPSS2 transcripts in a series of organs could explain the viral manifestation in these tissues. Recent studies, for example, have shown SARS-CoV-2 in stool samples from infected individuals and significant effects of the virus across tissues.

### COVID-19 ARDS and Increased Human Leukocyte Elastase Activity

The pathophysiology of ARDS in COVID-19 has not been elucidated yet. Without being bound by any particular mechanism, Applicant hypothesizes that the viral infection leads to an inflammatory host response, especially in the lung, which leads to sequestration and activation of granulocytes in the lower respiratory tract and the alveoli. Significant evidence exists that HLE is responsible for the depletion of at least one of the surfactant proteins, surfactant protein D (SP-D) during inflammation of the lung. Surfactant proteins A, B, C and D are part of the surfactant which is produced by type II alveolar cells and functions to lower surface tension in the interphase between the liquid and air phases at the alveoli. It is hypothesized that an increase in the activity of HLE at the alveoli can lead to the rapid and catastrophic deterioration of respiratory function in patients with ARDS secondary to COVID-19 infection. If true, this theory presents a number of potential therapeutic opportunities including some that are immediate.

### Alpha 1 Antitrypsin Deficiency (AAT) Allele Carriers and Risk for COVID-19 ARDS

Individuals who are carriers of genetic polymorphisms that lead to AAT would be predicted to be at increased risk of ARDS associated with COVID-19 infection. Given that the rapid pandemic has overwhelmed health care systems, it is important to identify individuals with the highest risk of mortality. It has been discussed that older individuals and individuals with underlying medical conditions are at higher risk for severe complications and death. However, differences in outcomes exist in this population and additionally, with the expansion of the infected population, it is now apparent that younger people without any apparent underlying conditions are becoming severely ill and some of them are dying of acute respiratory failure. It is imperative that the necessary epidemiological analyses be conducted rapidly, and the data broadly shared, in order to better assess and identify individuals at risk who may require additional and urgent interventions.

Europe has been the epicenter of the COVID-19 epidemic which is associated with a high prevalence of ARDS and associated mortality. The prevalence of AAT alleles reported in the literature as well as the emerging COVID-19 mortality data suggests a trend of higher mortality in populations that have higher allele frequency for either the S or the Z AAT alleles. In the review by Blanco et al., they reported the following: “In Europe, the mean SZ prevalence by regions (from the highest to the lowest) was as follows: Southern Europe, 1 SZ per 483 subjects (1:483); Western Europe, 1:581; Northern Europe, 1:1,492; Central Europe, 1:1,712; and Eastern Europe, 1:11,81.8.” Therefore, higher COVID-19 mortality would be expected in Southern Europe and lower mortality in Central and Eastern Europe.

The Pi*SZ genotype is more prevalent in Southern Europe and less prevalent in Central Europe. It should also be noted that the Italian peninsula shows higher prevalence in the North as compared to the South where the prevalence is very low.

The accumulating data for mortality during COVID-19 infections (March 31, 2020) shows a correlation between mortality rate (defined as deaths/cases confirmed) and the reported prevalence of the Pi*SZ A1AT gene allele by country. For robustness Applicant has included data only for the nine EU countries that had, at the time of filing, reported over 10,000 confirmed cases. Results of this correlation analysis are shown in Table 5. A significant correlation of R=0.66 (pvalue=0.05) was observed using all nine country mortality rates and Pi*SZ prevalence. From Blanco et al (2017), we observe that there is a significant difference in prevalence of the Pi*SZ genotype between North Italy (higher) and South Italy (lower). We have therefore reanalyzed the data this time excluding Italy. In this analysis also shown in Table 5, (excluding Italy), the correlation between mortality rate and Pi*SZ prevalence is even stronger R=0.88 (pvalue=0.003) Table 5.

TABLE 5
Correlation between COVID-19 mortality and Pi*SZ genotype

| Country | Cases | Deaths | Deaths/Cases | Pi*SZ (1 in:) |
|---|---|---|---|---|
| Italy | 105792 | 12428 | 0.117475802 | 967 |
| Spain | 94417 | 8269 | 0.087579567 | 278 |
| France | 44550 | 3024 | 0.067878788 | 413 |
| Germany | 68180 | 682 | 0.010002933 | 1337 |
| Switzerland | 16186 | 395 | 0.024403806 | 1152 |
| Belgium | 12775 | 705 | 0.05518591 | 551 |
| Austria | 10109 | 128 | 0.012661984 | 1680 |
| Netherlands | 12595 | 1039 | 0.082493053 | 617 |
| UK | 25150 | 1789 | 0.071133201 | 900 |
| Pearson's Correlation | | | | |
| All Countries | | R = 0.66 | pvalue = 0.05 | |
| (n = 9) | | | | |
| Excluding Italy | | R = 0.88 | pvalue = 0.003 | |
| (n = 8) | | | | |

These results suggest that a Pi*SZ genotype status may be a risk factor for COVID-19 ARDS and resulting mortality. With further confirmation, this observation may suggest that a different therapeutic approach is instituted for patients with COVID-19 infection and the Pi*SZ genotype that can include aggressive supportive therapy and the institution of elastase activity reducing treatments that may include small molecule inhibitors of the enzyme and/or supplementation of the A 1AT activity.

### Human Leukocyte Elastase (HLE) Inhibitors in the Treatment of COVID-19 ARDS

There has been interest in the development of HLE inhibitors for the treatment of emphysema and the treatment of patients with inherited forms of alpha 1 antitrypsin (A1AT) deficiency.

### Sivelestat

Sivelestat is currently available in Japan and Korea for the treatment of Acute Lung Injury (ALI) including ARDS. A number of clinical studies Aikawa and Kawasaki support the therapeutic utility of sivelestat in ARDS. Nonetheless the magnitude of the clinical benefit is still debated. In one Phase III study in 230 ventilated patients with ALI, sivelestat reduced duration of mechanical ventilation and shortened ICU stay, however, no significant effect was seen on the 30-day survival rate. In another study of 492 patients there was no effect on the ventilator free days or 28-day all-cause mortality. However, in a postmarketing study designed to reevaluate the efficacy of sivelestat in 404 ALI patients and 177 controls, sivelestat significantly improved the number of ventilator free days. While the differences in the results of these studies may have been due to differences in the study population and the design of the study, sivelestat is currently widely used in Japan and Korea in the ICU setting for patients with ARDS.

### Zemaira®

Zemaira® is an alpha-proteinase inhibitor (A 1-Pi) approved by the U.S. Food and Drug Administration and is indicated for chronic augmentation and maintenance therapy in adults with A1AT deficiency and clinical evidence of emphysema. Zemaira® is not approved for lung disease patients for whom severe A1AT deficiency has not been established.

### Alvelestat (MPH996)

Alvelestat is an experimental leukocyte elastase inhibitor under development in the US for the treatment of patients with alpha 1 antitrypsin deficiency of Pi*ZZ, Pi*SZ or Pi*Null/Null genotype. According to NCATS, “the drug's clinical profile suggests that it will be well tolerated with few, if any, side effects, and the existence of simple methods that can indirectly measure its activity in vivo.”

Unbalanced over-activity of the human leukocyte elastase is likely to play a role in the production and progression of the symptoms of acute respiratory distress in COVID-19 patients. Administration of small molecule, peptide or protein inhibitors that act to reduce the activity of HLE, including by direct interference with its enzymatic activity or down regulation of its coding neutrophil elastase gene (ELANE), is likely to be of therapeutic value to critically ill COVID-19 patients and as such it would be worth studying in controlled clinical trials. Moreover, Applicant's observation of higher mortality rate among COVID-19 patients with the Pi*SZ genotype, if confirmed, may suggest specific therapeutic options and treatment plan for these patients.

### TMPRSS2

A TMPRSS2 single nucleotide polymorphism, rs8134378, has been shown to reduce binding and transactivation by the androgen receptor. It is therefore possible that TMPRSS2 protease levels on the cell surface may vary depending on androgen levels. Other genetic sequence variations may also lead to variability in TMPRSS2 expression on cell membrane surfaces.

For example, a number of expression quantitative trait loci (eQTL) are reported in the Genotype-Tissue Expression (GTEx) database that are specifically associated with variable expression of the TMPRSS2 gene. These include rs8134657, rs8134378, rs6517673, rs9979885, rs9984523, rs9978587, rs28360562, rs34205539, rs1041449, and rs3498323. In each of these nine polymorphisms, carriers of the minor allele were associated with lower TMPRSS2 expression, suggesting higher expression among carriers of the alternative major allele.

Given these findings and the role of TMPRSS2 as a cellular receptor for SARS-CoV-2, antiandrogen therapy may lower levels of TMPRSS2, thereby decreasing the ability of the virus to enter human cells, lowering viral loads, and leading to better clinical outcomes.

In practicing the various embodiments of the invention, an antiandrogenic agent, i.e., a medicine that blocks the action of androgens or male sex hormones, such as testosterone, may be employed. Suitable antiandrogenic agents include, for example, androgen receptor (AR) antagonists, i.e., medicines that directly block the effects of androgens. Examples of AR antagonists, as noted above, include steroidal antagonists—such as cyproterone acetate, megestrol acetate, chlormadinone acetate, spironolactone, oxendolone, and osaterone acetate, dienogest, drospirenone, medrogestone, nomegestrol acetate, promegestone, and trimegestone, as well as non-steroidal antagonists—such as flutamide, bicalutamide, nilutamide, topilutamide, enzalutamide, and apalutamide.

Other suitable antiandrogenic agents include androgen synthesis inhibitors, i.e., medicines that act to lower androgen levels. Such agents include CYP17A1 inhibitors, such as ketoconazole, abiraterone acetate, and seviteronel, as well as aminoglutethimide, a CYP11A1 inhibitor. Other androgen synthesis inhibitors include 5α-reductase inhibitors, such as finasteride, dutasteride, epristeride, alfatradiol, and Serenoa repens (saw palmetto) extract.

Still other antiandrogenic agents include antigonadotropins, i.e., medicines that, like androgen synthesis inhibitors, act to lower androgen levels. These include the gonadotropin-releasing hormone (GnRH) modulators, such as cetrorelix; progestogens, such as allylestrenol, chlormadinone acetate, cyproterone acetate, gestonorone caproate, hydroxyprogesterone caproate, medroxyprogesterone acetate, megestrol acetate, osaterone acetate, and oxendolone; and estrogens, such as estradiol, estradiol esters, ethinylestradiol, conjugated estrogens, and diethylstilbestrol.

Other antiandrogenic agents may be employed in addition to those described and exemplified above, as are known in the art.

In practicing aspects of the invention, one or more antiandrogenic agents, including one or more of the agents noted above, are administered to an individual in an amount sufficient to reduce TMPRSS2 activity in the individual. Such reduced TMPRSS2 activity may result from direct blocking of androgens (such as through use of an AR antagonist), reduced androgen production or synthesis (such as through the use of androgen synthesis inhibitors or antigonadotropins), or both. In some embodiments of the invention, both an AR antagonist and an androgen synthesis inhibitor or antigonadotropin are employed, including any number of or combination of the agents set out above.

In the practice of the method of the present invention, an individual is selected for treatment based upon manifestations of ARDS. ARDS results when fluid builds up in the alveoli of the lungs, resulting in oxygen repletion in the bloodstream, depriving organs of the oxygen needed to function. Severe shortness of breath is the main symptom of ARDS and can develops within hours to days following a precipitating infection, such as a diagnosed or suspected coronavirus 2 (SARS-CoV-2) infection. ARDS, once the syndrome develops, is often fatal. The risk of death increases with age and co-morbidities. ARDS can produce lasting damage to the lungs. The diagnosis of ARDS is accomplished using established diagnostic criteria known in the art, as is the determination of whether the individual is suffering from a coronavirus 2 (SARS-CoV-2) infection or is suspected to have such an infection based on symptoms or possible exposure to the virus. Diagnostic testing for the infection is known in the art.

In addition, in the practice of the present invention, the amount of the antiandrogenic agent administered to the individual is determined by the condition of the patient, the potency of the agent, the age and weight of the patient and other criteria known in the art for the producing the desired antiandrogenic effect. The treatment according to the present method is initiated at any point following a determination that the individual to be treated has a known or suspected coronavirus 2 (SARS-CoV-2) infection that has produced manifestations of ARDS or that, for an asymptomatic patient, that may result in ARDS. For example, patients at elevated risk for developing ARDS may be administered an antiandrogenic agent prophylactically prior to the diagnosis of ARDS. Such patients may include those of advanced age (e.g., over the age of 60 or 65) and those with co-morbidities, e.g., hypertension, diabetes, cardiovascular disease, asthma, or disorders of the immune system including immunosuppressed individuals.

Treatment of an individual in accordance with the present method can be sustained until the desired amelioration of one or more symptoms of ARDS is observed or sustained until the ARDS has fully resolved or so long as needed for the individual to regain lung function that may have been compromised as a result of the syndrome. Accordingly, treatment of the individual may be sustained for a period of days to weeks from the initiation of therapy or, if needed for months.

### ACES2

Natural genetic variation in ACE2 may affect individual susceptibility to SARS-CoV-2 infection and the propagation capacity of the virus. Specifically, ACE2 genotype-tissue analysis points to rather rare population frequencies of potential variants that may constitute susceptibility or resilience to infection of SARS-CoV-2 in certain individuals. There are also age-related differences in the expression of ACE2 relative to ACE. The ACE2/ACE ratio is much higher among younger individuals. In addition, because the human ACE2 gene is located on the X chromosome, males who carry rare ACE2 coding variants will be hemizygous, expressing only those rare variants in all ACE2-expressing cells. Females carrying rare ACE2 coding variants, on the other hand, are significantly more likely to be heterozygous and will typically express those rare ACE2 variants in a mosaic distribution determined by early X-inactivation events.

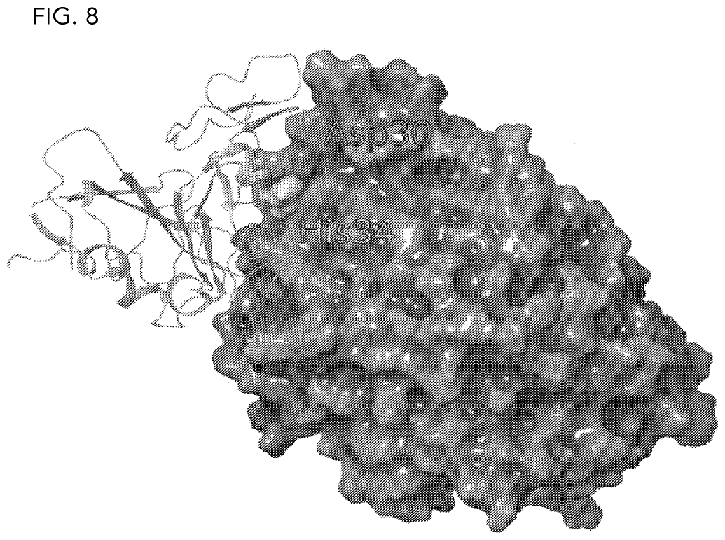

Comparing the human ACE2 amino acid sequence (SEQ ID 12) to those of other animals (chicken, pig, dog, and cat) as well as to their reported susceptibilities to SARS-CoV-2 infection, and focusing on functional, contact amino acid residues, certain amino acids are likely important to viral entry. One amino acid in particular, the histidine at position 34 (His34; shown in blue in FIG. 8), appears critical for viral entry across all species. Other ACE2 amino acids likely important to SARS-CoV-2 viral entry include ASP³⁰ (shown in red in FIG. 8), Tyr⁴¹, Gln⁴², LyS³⁵³, and Arg³⁵⁷.

It has been suggested that the His³⁴ of the ACE2 sequence likely engages in hydrogen bonding with the tyrosine at position 453 (Tyr⁴⁵³), located within the RBD of the S protein sequence (SEQ ID 2). ASP³⁰ of ACE2 is believed to similarly bond with Lys⁴¹⁷ of the RBD, as are Tyr⁴¹, Gln⁴², LyS³⁵³, and Arg³⁵⁷ of ACE2 with Gln⁴⁹⁸, Thr⁵⁰⁰, and Asn⁵⁰¹ of the RBD.

Conducting a in silico chemical library screen permits the identification of small molecules that may be capable of binding to or masking binding to any of the amino acids of the ACE2 or RBD sequences. Such binding or masking affords a mechanism for the inhibition of SARS-CoV-2 viral entry, either to treat or prevent infection.

The glide docking protocol is first applied. This includes the calculation of a GlideScore for candidate molecules to predict the binding of ACE2 and the RBD. GlideScores are calculated using the Glide software available from Schrodinger, LLC. The components and use of GlideScore calculations are known in the art, specifically the methodology described and marketed by Schrodinger, LLC in, for example, their Glide 6.7 User Manual, which is hereby incorporated herein as though fully set forth.

The docking score (GlideScore) is an empirical scoring function designed to maximize separation of compounds with strong binding affinity from those with little to no binding ability. As an empirical scoring function, it is comprised of terms that account for the physics of the binding process including a lipophilic-lipophilic term, hydrogen bond terms, a rotatable bond penalty, and contributions from protein-ligand coulomb-vdW energies. The lower the docking score the more optimal the docking. Candidate molecules are assessed for actual interactions at the docking site (also how the molecule anchors), hbond score (the hbond term will be lower if the hydrogen bonds are more optimal, for example at closer distance) and ligand efficiency (normalized version of the glide score (gscore) divided by a number of heavy atoms).

Next, a determination of pharmacokinetically relevant molecular descriptors of the candidate molecules is made. And, finally, molecular dynamics simulations are conducted to validate the stability of docked binding modes. A total of approximately 11,000 non-redundant candidate molecules are analyzed.

Of the candidate molecules screened, the 10 with the lowest (i.e., most favorable to binding) docking score are shown below in Table 6.

TABLE 6

| | | | glide | | glide | |
|---|---|---|---|---|---|---|
| | | | rotatable | docking | ligand | glide |
| DrugID | Drug | CAS ID | bonds | score | efficiency | hbond |
| ZINC000098052516 | AY-NH₂ | 352017-71-1 | 24 | −8.541 | −0.174 | −0.916 |
| ZINC000008214766 | Nicotinamide | 53-84-9 | 15 | −7.979 | −0.181 | −0.852 |
| | adenine | | | | | |
| | dinucleotide NAD+ | | | | | |
| ZINC000001542931 | Reproterol | 54063-54-6 | 10 | −7.71 | −0.275 | −1.169 |
| ZINC000245219534 | Thymopentin | 69558-55-0 | 28 | −7.645 | −0.159 | −1.423 |
| ZINC000009228252 | CGS 21680 HCl | 124431-80-7 | 13 | −7.57 | −0.21 | −1.084 |
| ZINC000008215403 | Disodium NADH | 606-68-8 | 15 | −7.467 | −0.17 | −1.214 |
| ZINC000098052511 | Nociceptin (1-7) | 178249-42-8 | 25 | −7.249 | −0.154 | −0.83 |
| ZINC000257482989 | Mupirocin | 12650-69-0 | 20 | −7.078 | −0.202 | −1.124 |
| ZINC000026468553 | SLIGRL-NH2 | 171436-38-7 | 29 | −6.973 | −0.152 | −0.949 |
| ZINC000000001872 | Oxiniacic Acid | 2398-81-4 | 1 | −6.948 | −0.695 | −0.16 |

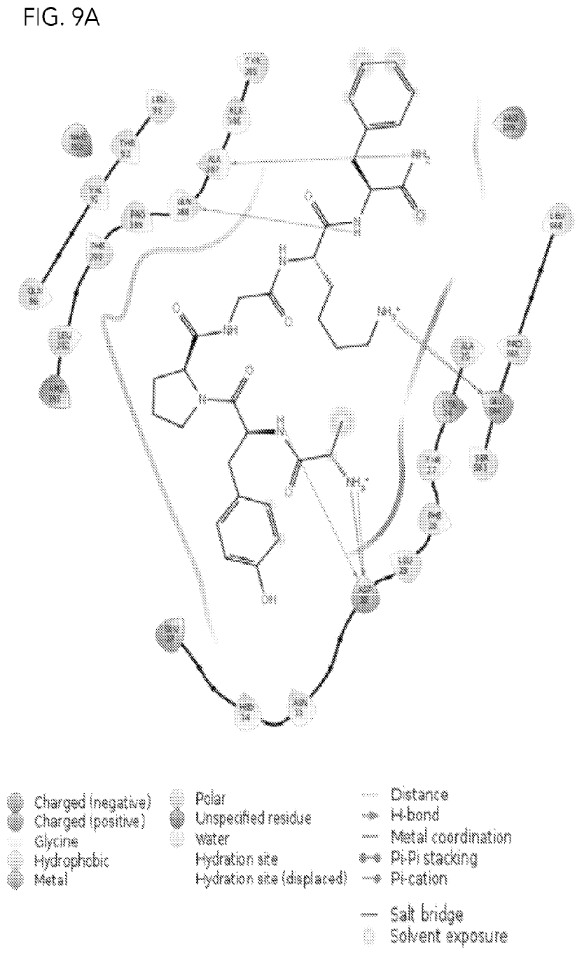

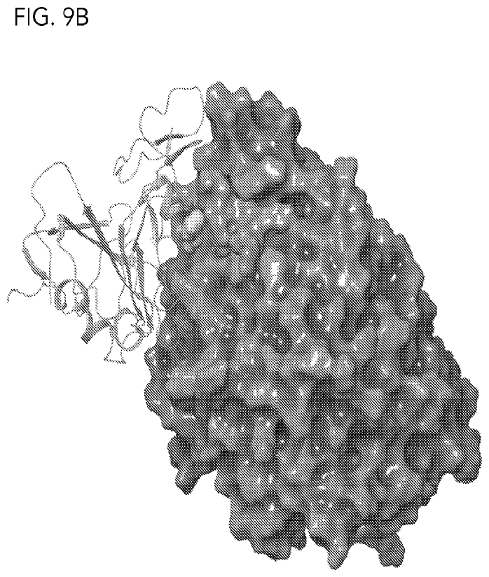

AY-NH₂ is a selective PAR4 receptor agonist peptide (H-Ala-Tyr-Pro-Gly-Lys-Phe-NH₂) and yields the most favorable docking score. FIG. 9A is a graphical depiction of the AY-NH₂ compound annotated according to the determinations described above with respect to predicted interactions of the compound with the relevant amino acids of ACE2. These include predicted hydrogen bonding with the ACE2 Asp³⁰, Ala³⁸⁷, Gln³⁸⁸, and Glu⁵⁶⁴ amino acids. FIG. 9B is a three-dimensional representation of AY-NH₂ bound to ACE2 as predicted above. Bound as such, binding by the SARS-CoV-2 S protein RBD to the His³⁴ (blue) or ASP³⁰ (red) of ACE2 is effectively blocked by NY-HN₂ (grey).

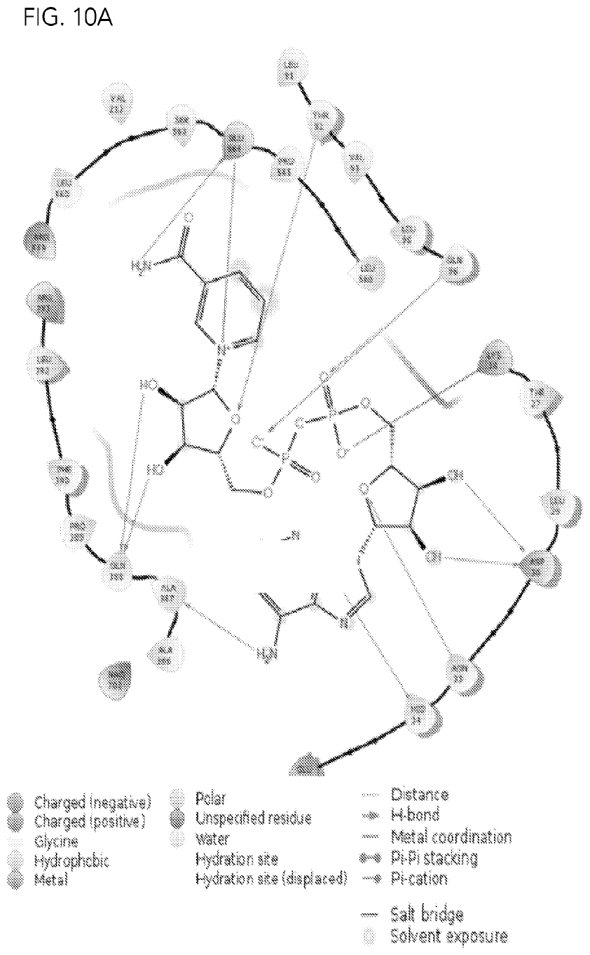

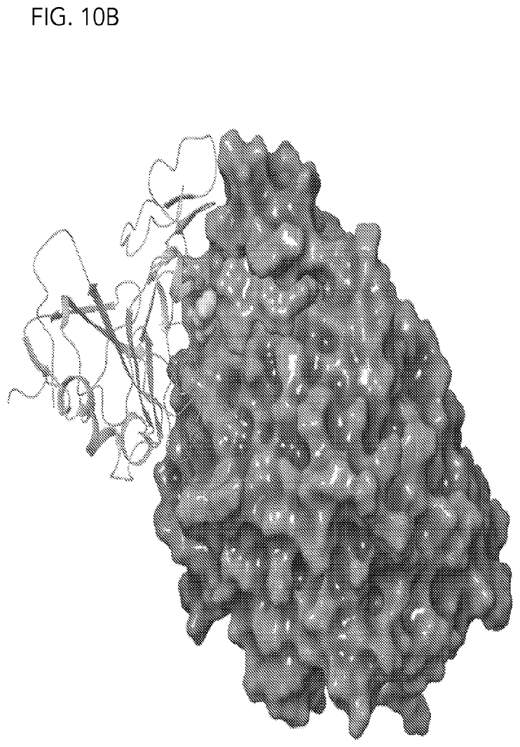

NAD⁺ (oxidized nicotinamide adenine dinucleotide), a coenzyme involved in many metabolic reactions, yields the second most favorable docking score. NAD⁺ plasma levels have been reported to significantly decline with age. Recent studies indicate that SARS-CoV-2 infection of cell lines significantly dysregulates the NAD⁺ pathway with respect to NAD⁺ synthesis and utilization. FIG. 10A is a graphical depiction of the NAD⁺ compound similarly annotated according to the determinations described above with respect to predicted interactions of the compound with the relevant amino acids of ACE2. FIG. 10B is a three-dimensional representation of NAD⁺ bound to ACE2 as predicted. Bound as such, binding by the SARS-CoV-2 S protein RBD to the His³⁴ (blue) or ASP³⁰ (red) of ACE2 is effectively blocked by NAD⁺ (grey).

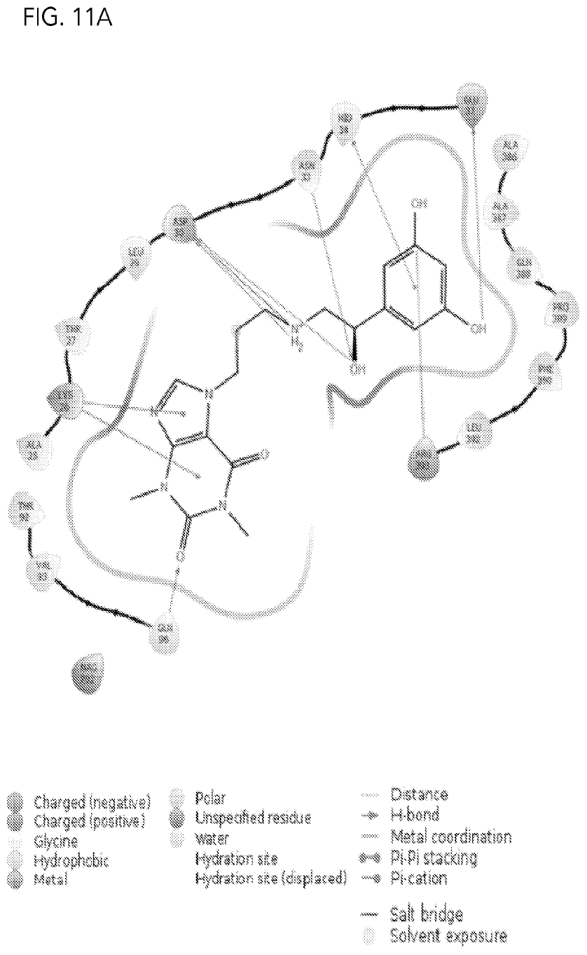

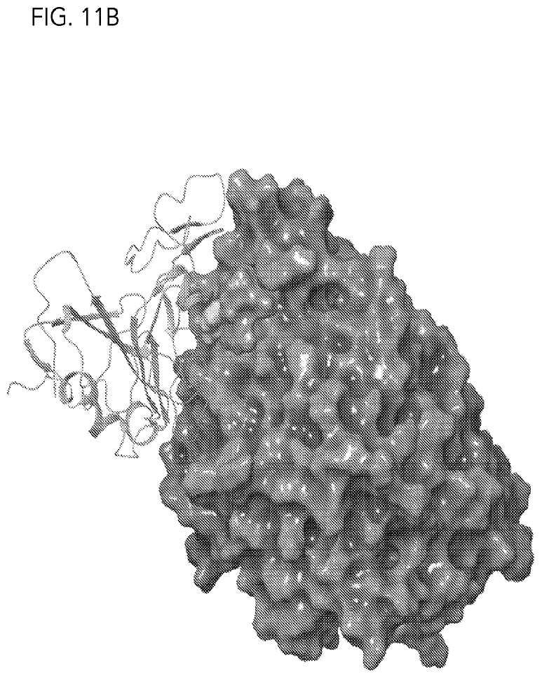

Reproterol (7-[3-[[2-(3,5-dihydroxyphenyl)-2-hydroxyethyl]amino]propyl]-1,3-dimethylpurine-2,6-dione) is a short-acting β₂ adrenoreceptor agonist approved for use in the treatment of asthma. It yields the third most favorable docking score. FIG. 11A is a graphical depiction of the reproterol compound annotated according to the determinations described above with respect to predicted interactions of the compound with the relevant amino acids of ACE2. FIG. 11B is a three-dimensional representation of reproterol bound to ACE2 as predicted. Bound as such, binding by the SARS-CoV-2 S protein RBD to the His³⁴ (blue) or ASP³⁰ (red) of ACE2 is effectively blocked by reproterol (grey).

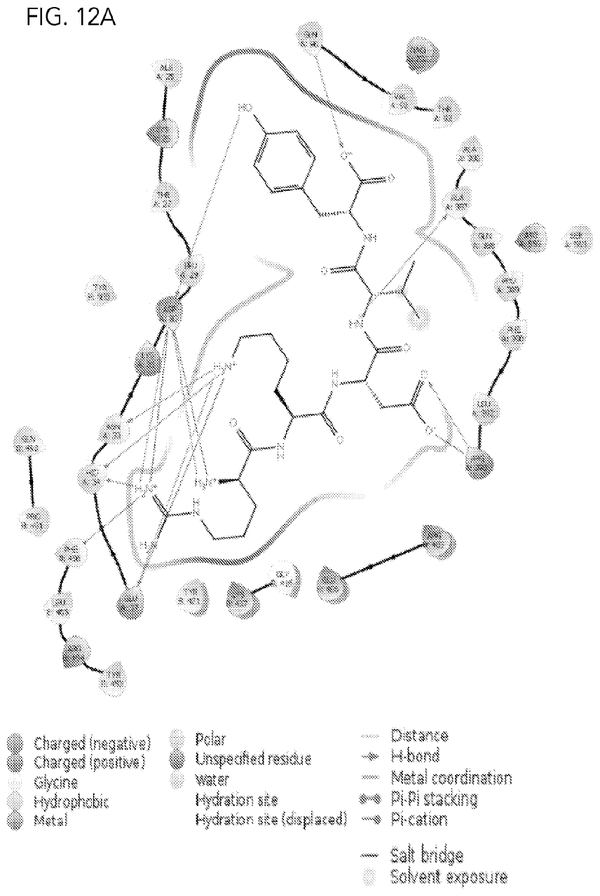

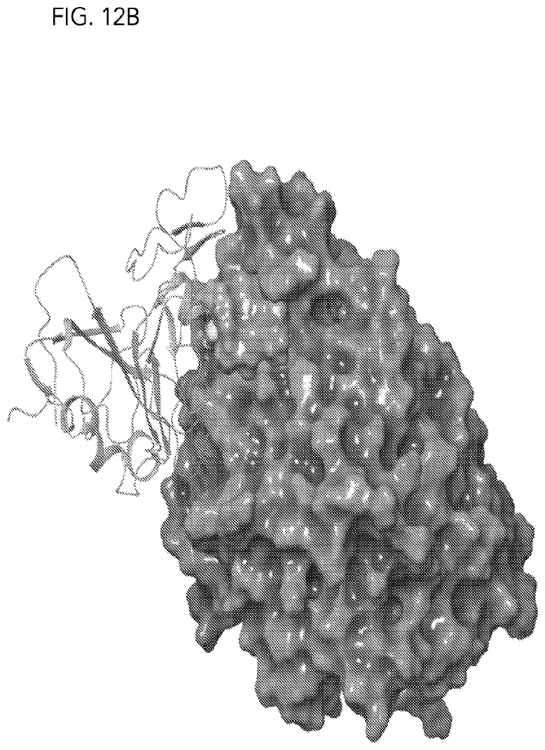

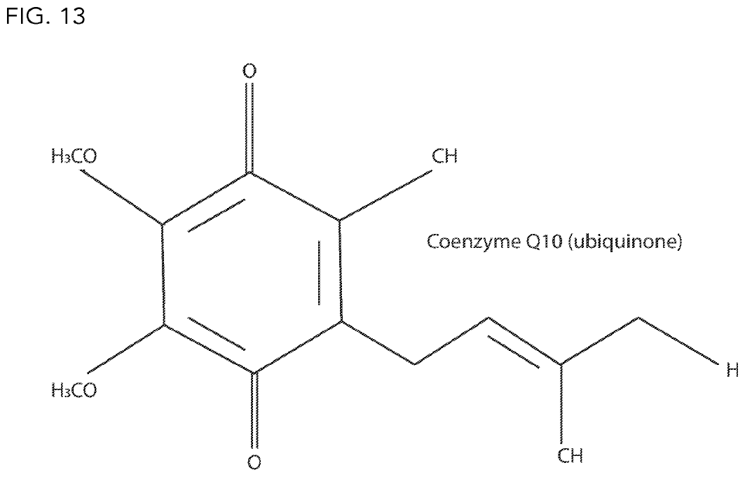

Thymopentin (H-Arg-Lys-Asp-Val-Tyr-OH) is a synthetic pentapeptide used to enhance the production of thymic T cells. It yields the fourth most favorable docking score. FIG. 12A is a graphical depiction of the thymopentin compound annotated according to the determinations described above with respect to predicted interactions of the compound with the relevant amino acids of ACE2. FIG. 12B is a three-dimensional representation of thymopentin bound to ACE2 as predicted. Bound as such, binding by the SARS-CoV-2 S protein RBD to the His³⁴ (blue) or ASP³⁰ (red) of ACE2 is effectively blocked by thymopentin (grey).

The other compounds of Table 6 yielded lower docking scores but are capable of acting to inhibit binding at His34 and other of the ACE2 amino acids noted above, thereby inhibiting SARS-CoV-2 infection.

CGS 21680 HCl (2-p-(2-Carboxyethyl)phenethylamino-5′-N-ethylcarboxamidoadenosine hydrochloride) is a selective adenosine A2A-R agonist.

Disodium NADH (reduced disodium nicotinamide adenine dinucleotide) is a coenzyme of a large number of oxidoreductases.

Nociceptin (1-7) (nociception fragment 1-7; H-Phe-Gly-Gly-Phe-Thr-Gly-Ala-OH) is an active metabolite of nociception.

Mupirocin (9-[(E)-4-[(2S,3R,4R,5S)-3,4-dihydroxy-5-[[(2S,3S)-3-[(2S,3S)-3-hydroxybutan-2-yl]oxiran-2-yl]methyl]oxan-2-yl]-3-methylbut-2-enoyl]oxynonanoic acid; pseudomonic acid) is a naturally-occurring antibiotic currently used topically to treat impetigo and other staph infections of the skin. It has also been used in the treatment of methicillin-resistant S. aureus (MRSA) infections.

SLIGRL-NH₂ (H-Ser-Leu-Ile-Gly-Arg-Leu-NH₂) is a peptide derived from the N-terminus of protease-activated receptor-2 (PAR2) and acts as a PAR2 agonist.

Oxiniacic acid (3-pyridinecarboxylic acid 1-oxide; nicotinic acid 1-oxide) is a nicotinic acid derivative with hypolipidemic activity.

In the practice of the method of the present invention, an individual is selected for treatment based upon manifestations of symptoms associated with SARS-CoV-2 infection, including acute respiratory distress syndrome (ARDS), or the individual's risk of SARS-CoV-2 infection. Diagnostic testing for SARS-CoV-2 infection is known in the art.

Of particular concern in determining whether an individual is to be treated is the manifestation of ARDS, which results when fluid builds up in the alveoli of the lungs, resulting in oxygen repletion in the bloodstream, depriving organs of the oxygen needed to function. Severe shortness of breath is the main symptom of ARDS and can develop within hours to days following a precipitating infection, such as a diagnosed or suspected SARS-CoV-2 infection. ARDS, once the syndrome develops, is often fatal. The risk of death increases with age and co-morbidities. ARDS can produce lasting damage to the lungs. The diagnosis of ARDS is accomplished using established diagnostic criteria known in the art, as is the determination of whether the individual is suffering from a SARS-CoV-2 infection or is suspected to have such an infection based on symptoms or possible exposure to the virus.

In addition, in the practice of the present invention, the amount of the treating agent (e.g., AY-NH₂, NAD⁺, Reproterol, Thymopentin, CGS 21680 HCl, disodium NADH, Nociceptin (1-7), Mupirocin, SLIGRL-NH₂, or oxiniacic acid) administered to the individual is determined by the condition of the patient, the potency of the agent, the age and weight of the patient and other criteria known in the art.

Treatment according to the present method may be initiated at any point following a determination that the individual to be treated has a known or suspected SARS-CoV-2 infection that has produced manifestations of ARDS or, for an asymptomatic patient, that may result in ARDS. For example, patients at elevated risk for developing ARDS may be administered a treating agent prophylactically prior to the diagnosis of ARDS. Such patients may include those of advanced age (e.g., over the age of 60 or 65) and those with co-morbidities (e.g., hypertension, diabetes, cardiovascular disease, asthma, or disorders of the immune system including immunosuppressed individuals).

### Coenzyme Q10 and COVID-19

A potential association may exist between reduced levels of CoQ10 and the population of people most severely affected by COVID-19. The causative mechanisms of creating a susceptibility to severe illness remain unclear, though they could be a result of a reduced ability to either prevent oxidative stress, attenuate coagulation, mitigate a hyper-immune response, or inhibit viral replication of entry directly. The deficiencies associated with disease may involve other antioxidants such as Vitamin C and E. Large studies should measure CoQ10 levels along with vitamins and lipids of infected people with COVID-19 at the time of presentation to examine whether correlations predict clinical outcomes and correlate with the levels of inflammatory cytokines and molecules such as IL-2, IL-6, TNF-alpha and D-dimer. Clinical outcomes of COVID-19 in individuals with genetically predisposed CoQ10 deficiency should also be examined. Given the complexity of SARS-CoV2 infection and heterogeneity in disease presentation, the reasons for severe illness are likely multifactorial. CoQ10 may serve as a correlated marker in severe illness, and potentially as a causative agent for susceptibility to worse clinical outcomes.

If an association is confirmed, a causative mechanism could further be explored and CoQ10 may potentially offer a protective therapy in the future. Dosing to replenish levels of CoQ10 in deficiency could begin between 100 mg to 200 mg daily to have physiological impact. As it is the case with many disease states, more impactful benefits can be made with prophylaxis. If lower levels of CoQ10 are correlated with severe COVID-19 illness, supplementation of deficient individuals could potentially offer a therapeutic solution to reduce the burden of disease and potentially improve the state of this pandemic.

### Severe COVID-19 and FYCO1

The genomes of 80 COVID-19 patients (68% male, 32% female; ages 35-87) exhibiting severe symptoms were analyzed and compared to the genomes of 1,876 individuals from a 2000 genome-wide association study (GWAS). The results of this comparison not only confirm the previously reported association between severe COVID-19 infection and the six-gene Neanderthal haplotype, but also point to three mutations within the FYCO1 gene and severe COVID-19 infection, as well as their association with the rs73064425 SNP within the LZTFL1 gene. This more discrete haplotype showed the strongest association with severe COVID-19 infection and may be useful in predicting and diagnosing severe COVID-19 infection.

The LZTFL1 SNP, rs73064425, a C-to-T variant, has a reported minor allele frequency (MAF) of 0.05. This value was consistent with that found within the 2000 control genotypes. Among the COVID-19 patients, however, the MAF was 0.17. This SNP was in strong linkage disequilibrium with each of the three FYCO1 SNPS. These FYCO1 SNPs, two of which occur in the same codon represent three coding mutations, cause amino acid substitutions in the resulting mRNA.

Of the newly associated FYCO1 SNPs, the first, rs13079478, is a G/T variant resulting in an amino acid substitution of aspartic acid for asparagine; the second, rs13059238, is a T/C variant in the same codon as rs13079478, but results in an amino acid substitution of lysine for asparagine; and the third, rs33910087, is a G/A variant resulting in an amino acid substitution of cysteine for arginine.

Table 7 below shows the MAF for each FYCO1 SNP within the COVID-19 population and the 2000 control population.

TABLE 7

| | | | Control | COVID-19 | |
|---|---|---|---|---|---|
| SNP | WT | MA | MAF | MAF | P value |
| rs73064425 | C | T | 0.0533 | 0.1696 | 1.19 × 10⁻⁵ |
| rs13079478 | G | T | 0.07809 | 0.2143 | 7.84 × 10⁻⁶ |
| rs13059238 | T | C | 0.08316 | 0.2232 | 7.01 × 10⁻⁶ |
| rs33910087 | G | A | 0.08076 | 0.2143 | 1.34 × 10⁻⁵ |

As can be seen in Table 7, the frequency of the minor allele at each of the FYCO1 and LZTFL1 SNPs is significantly higher within the COVID-19 population. This offers a useful way of predicting whether an individual is predisposed to severe COVID-19 symptoms in the event of exposure, as well as a valuable treatment tool in the treatment of COVID-19 patients, enabling identification of those patients more likely to experience severe COVID-19 symptoms and earlier treatment of those patients for such symptoms.

### The FYCO1 Gene

The FYCO1 gene encodes a protein involved in vesicle transport and autophagy. It has been suggested to be a key mediator linking endoplasmic reticulum-derived double membrane vesicle, the primary replication site for coronaviruses, with the microtubule network.

Through its LC3-interacting region (LIR) motif, FYCO1 has also been shown to be important for the fusion of autophagosomes with lysosomes. FYCO1 dimerizes via the CC region, interacts with PI3P via its FYVE domain, and forms a complex with Rab7 via a part of the CC region located in front of the FYVE domain. Specifically, FYCO1 was shown to act as a Rab7 effector that binds to LC3 and PI3P to mediate microtubule plus end-directed vesicle transport. The depletion of Rab7 inhibits maturation of late endosomes/multivesicular bodies (MVBs) and leads to reduced lysosome numbers in cells. FYCO1 also mediates clearance of a-synuclein aggregates.

A genome-scale CRISPR loss-of-function screen in human alveolar basal epithelial carcinoma cells identified genes whose loss enabled resistance to SARS-CoV-2 infection. The loss of RAB7A reduces viral entry/egress by sequestering the ACE2 receptor inside cells. Depletion of FYCO1 antibodies against the N-terminus of LC3 blocks the subcellular redistribution of autophagosomes. Some rare FYCO1 variants containing missense mutations in the LIR domain are associated with inclusion body myositis, a disease characterized by impaired autophagic degradation.

Gain-of-function variants in FYCO1 confer a greater risk of severe COVID-19 infection and may confer a similarly greater risk with respect to other betacoronaviruses. As such, downregulation of FYCO1 would offer protection against such severe infection, offering a potential therapeutic against COVID-19.

A high-throughput gene expression analysis identified compounds capable of downregulating FYCO1. A total of 466 compounds from 14 therapeutic classes were subjected to such analysis using human retinal pigment epithelial cell line ARPE-19 and gene expression changes collected across 12,490 genes. The effect of all 466 compounds on FYCO1 expression is greatest for the four compounds shown in Table 8.

TABLE 8

| Compound | Treated | Control | Difference |
|---|---|---|---|
| indomethacin | 4.949 | 6.488 | −1.539 |
| primidone | 6.4453 | 7.9412 | −1.4959 |
| triprolidine hydrochloride | 6.4963 | 7.9898 | −1.4935 |
| baclofen | 6.7222 | 7.9412 | −1.2190 |

Indomethacin, a non-steroidal anti-inflammatory, exhibits the greatest ability to inhibit or downregulate FYCO1 expression. Indomethacin is approved for the treatment of rheumatoid arthritis, ankylosing spondylitis, osteoarthritis, gouty arthritis, bursitis, and tendonitis. It may be administered orally, intravenously, or rectally. Oral dosages are typically 75-150 mg daily in up to four divided doses. Oral dosages are available in 20 mg, 25 mg, 40 mg, and 50 mg capsules, a 75 mg extended release capsule, and a 25 mg/5 mL oral suspension.

The terminology used herein is for the purpose of describing particular embodiments only and is not intended to be limiting of the disclosure. As used herein, the singular forms “a,” “an,” and “the” are intended to include the plural forms as well, unless expressly states otherwise or the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. “Optional” or “optionally” means that the subsequently described element, event, or circumstance may or may not occur, and that the description includes instances where the element, event, or circumstance occurs or is present and instances where it does not occur or is not present.

The corresponding structures, materials, acts, and equivalents of all means or step plus function elements in the claims are intended to include any structure, material, or act for performing a function in combination with other claimed elements as specifically claimed. The description of the present disclosure is presented for purposes of illustration and description but is not intended to be exhaustive or limited to the disclosure in the form disclosed. Many modifications and variations will be apparent to those of ordinary skill in the art without departing from the scope and spirit of the disclosure. Any embodiments chosen and described herein appear in order to best explain the principles of the disclosure and their practical application, and to enable others of ordinary skill in the art to understand the disclosure for various embodiments with various modifications as are suited to the particular use contemplated.

1. A method of identifying compounds useful in treating or preventing infection by severe acute respiratory syndrome coronavirus 2 (SARS-CoV-2), the method comprising:
screening at least one candidate compound for an ability to inhibit cleavage of a SARS-CoV-2 spike protein by a human protease at one or more target site.

2. The method of claim 1, further comprising:
determining, for the at least one candidate compound, a level of inhibition of host cell peptide cleavage.

3. The method of claim 1, wherein the one or more target site is selected from a group consisting of: between threonine at position 768 and glycine at position 769 of SEQ ID 1; and between arginine at position 815 and serine at position 816 of SEQ ID 1.

4. The method of claim 1, wherein the at least one candidate compound includes a plurality of compounds.

5. The method of claim 4, wherein screening includes screening the plurality of compounds in an assay.

6. The method of claim 1, wherein the ability to inhibit cleavage includes one or more measure selected from a group consisting of: binding of the at least one candidate compound with the human protease, binding of the at least one candidate compound to block a target site on the spike protein, a change of expression of the human protease, and a change of function of a lysosome in which the human protease is contained in a host cell.

7. The method of claim 1, further comprising:
determining whether the spike protein amino acid sequence includes one or more mutation from a wildtype sequence (SEQ ID 1), the one or more mutation being selected from a group consisting of: histidine at position 675 (SEQ ID 3), leucine at position 704 (SEQ ID 4), alanine at position 719 (SEQ ID 5), phenylalanine at position 752 (SEQ ID 6), leucine at position 765 (SEQ ID 7), leucine at position 772 (SEQ ID 8), glutamine at position 780 (SEQ ID 9), cysteine at position 797 (SEQ ID 10), and serine at position 812 (SEQ ID 11).

8-11. (canceled)

12. A method of treating or preventing infection by severe acute respiratory syndrome coronavirus 2 (SARS-CoV-2) in an individual, the method comprising:
administering to the individual amantadine in an amount sufficient to reduce lysosomal enzymatic activity in a cell of the individual.

13. The method of claim 12, wherein the amount of amantadine is sufficient to reduce expression of at least one gene in the individual, the at least one gene selected from a group consisting of: CTSL, AGA, BCL10, DC164, CLN5, CPQ, CTBS, CTSB, CTSH, CTSK, GALC, GJA1, GNS, LAMP1, LGMN, PCYOX1, PSAP, RAB38, RNASET2, SCARB2, STS, and MARCH3.

14. The method of claim 13, wherein the at least one gene is CTSL.

15. The method of claim 12, wherein the cell is located in respiratory tract tissue.

16. The method of claim 12, wherein the amount of amantadine is sufficient to decrease a viral load in the individual.

17. The method of claim 12, further comprising:
determining a genotype of the individual at the rs2378757 locus.

18. The method of claim 17, wherein the amount of amantadine administered to the individual is greater if the individual's rs2378757 genotype is determined to be AA than if the individual's rs2378757 genotype is determined to be AC or CC.

19. A method of decreasing a SARS-CoV-2 viral load in an individual infected therewith, the method comprising:
administering to the individual amantadine in an amount sufficient to reduce lysosomal enzymatic activity in a cell of the individual.

20. The method of claim 19, wherein the amount of amantadine is sufficient to reduce expression of at least one gene in the individual, the at least one gene selected from a group consisting of: CTSL, AGA, BCL10, DC164, CLN5, CPQ, CTBS, CTSB, CTSH, CTSK, GALC, GJA1, GNS, LAMP1, LGMN, PCYOX1, PSAP, RAB38, RNASET2, SCARB2, STS, and MARCH3.

21. The method of claim 20, wherein the at least one gene is CTSL.

22. The method of claim 19, wherein the cell is a lung tissue cell.

23. The method of claim 19, wherein the amount of amantadine is sufficient to decrease a viral load in the individual.

24. The method of claim 19, further comprising:
determining a genotype of the individual at the rs2378757 locus.

25-122. (canceled)
